(19) United States
(12) Patent Application Publication
(10) Pub. No.: US 2021/0008018 A1
(43) Pub. Date: Jan. 14, 2021

(54) DEUTERATED ANALOGS OF D-BETA-HYDROXYBUTYRIC ACID AND USES THEREOF

(71) Applicant: Concert Pharmaceuticals, Inc., Lexington, MA (US)

(72) Inventors: Roger D. Tung, Lexington, MA (US); I. Robert Silverman, Arlington, MA (US); Naeem Yusuff, Lexington, MA (US)

(21) Appl. No.: 17/026,413

(22) Filed: Sep. 21, 2020

Related U.S. Application Data
(63) Continuation-in-part of application No. PCT/US2019/023709, filed on Mar. 22, 2019.
(60) Provisional application No. 62/647,311, filed on Mar. 23, 2018.

(51) Int. Cl. A61K 31/19; A61K 9/00

(52) U.S. Cl. CPC A61K 31/19; A61K 9/0053

(57) ABSTRACT

This disclosure relates to deuterated D-β-hydroxybutyric acid (DBHB) of Formula I:

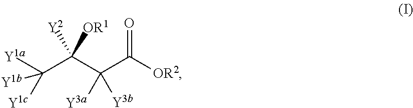

wherein each of the variables are defined herein, and pharmaceutically acceptable salts thereof, analogs and prodrugs thereof, pharmaceutical compositions thereof, and methods of use.

### CROSS-REFERENCES TO RELATED APPLICATIONS

This application is a continuation-in-part application of International Application No. PCT/US2019/023709, filed Mar. 22, 2019, which claims the benefit of the filing date of U.S. Provisional Application No. 62/647,311, filed Mar. 23, 2018. The entire contents of the aforementioned applications are incorporated herein by reference.

### BACKGROUND OF THE INVENTION

Exercise has long been known to have positive effects on mood, memory, and cognitive function. In recent years, much of the benefit associated with exercise has been attributed to increases in brain-derived neurotrophic factor, or BDNF. A member of the neurotrophin family of growth factors which are found in the brain and periphery, BDNF is known to be essential for maintaining healthy neurons and creating new neurons, thus protecting from neurological disease such as Alzheimer's disease and Parkinson's disease. See e.g. Servick, K., Science, Oct. 10, 2013, http://www.sciencemag.org/news/2013/10/how-exercise-beefs-brain. Additionally, decreased levels of BDNF are associated with depression (see Duman, R. S. and Monteggia, L. M., Biological Psychiatry, 2006, 59: 1116-1127), whereas increased levels promote improvement in cognitive ability and in symptoms of depression (see Marais, L. et al., Metabolic Brain Disease, 2009, 24: 587-597). As a vital component to normal brain function, inadequate levels of BDNF has been associated with many psychiatric disorders including major depressive disorder (MDD), schizophrenia, bipolar disorder, anxiety-related disorders, addiction, Rett Syndrome and eating disorders (see Autry, A. E. and Monteggia, L. M., Pharmacological Reviews, 2012, 64: 238-258).

D-β-hydroxybutyric acid (DBHB) is a ketone body formed in response to exercise, a high protein-low carbohydrate diet, or caloric restriction. DBHB has been shown to be an effective neuroprotective agent in preclinical models of Huntington's Disease (See Lim et al. PLoS ONE 6(9) 2011: e24620 (https://doi.org/10.1371/journal.pone.0024620); and Pollock et al., Molecular Therapy, 2016 May; 24(5): 965-977), Parkinson's Disease (PD) and Alzheimer's Disease (AD) (See Kashiwaya et al., PNAS, May 9, 2000; 97(10): 5440-5444; Kashiwaya et al., Neurobiol. Aging, 2012; 34: 1530-1539; and Tieu et al, The Journal of Clinical Investigation, 2003; 112:892-901). Recent studies have revealed that DBHB acts as an inhibitor of histone deacetylases HDAC2 and HDAC3, resulting in upregulation of BDNF transcription. See Sleiman et al. eLife 2016; 5:e15092, https://doi.org/10.7554/eLife.15092.

### SUMMARY OF THE INVENTION

DBHB has the following structure (A):

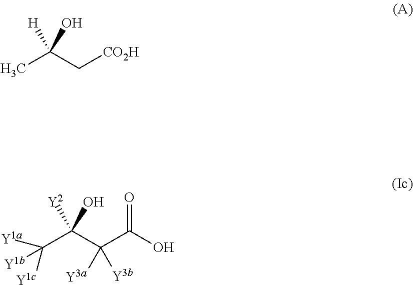

This invention relates to deuterated forms of DBHB shown by Formula (Ic) above where each Y is independently hydrogen or deuterium, pharmaceutically acceptable salts thereof, analogs and prodrugs thereof, pharmaceutical compositions thereof, and methods of use.

In one aspect, this invention relates to deuterated forms of DBHB, pharmaceutically acceptable salts thereof, analogs and prodrugs thereof, pharmaceutical compositions thereof, and methods of use.

In one aspect, the invention provides a pharmaceutical composition comprising a compound of Formula Ia:

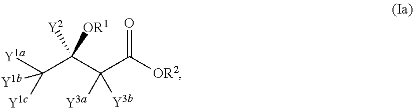

or a pharmaceutically acceptable salt thereof, wherein:

each of Y^{1a}, Y^{1b}, Y^{1c}, Y², Y^{3a} and Y^{3b} is independently H or D;

R¹ is H, D, —C(O)—C₁₋₆ alkyl or

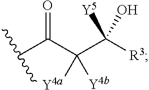

R² is H, D, —C₁₋₆ alkyl, —C₃₋₁₀ cycloalkyl, or

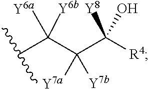

each of Y^{4a}, Y^{4b}, Y⁵, Y^{6a}, Y^{6b}, Y^{7a}, Y^{7b} and Y⁸, when present, is independently H or D;

each of R³ and R⁴, when present, is CH₃ or CD₃;

provided that at least one of Y^{1a}, Y^{1b}, Y^{1c}, Y², Y^{3a} and Y^{3b} is D; and further provided that when R¹ is H and R² is H, then at least one of Y², Y^{3a} and Y^{3b} is D;
and a pharmaceutically acceptable carrier.

In certain embodiments of the compositions of the invention, the compound of Formula Ia is a compound of Formula 2:

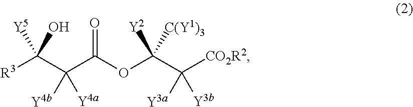

or
a pharmaceutically acceptable salt thereof, wherein:

each Y¹ is the same and is H or D;

each of Y², Y^{3a} and Y^{3b} is independently H or D;

each of Y^{4a}, Y^{4b} and Y⁵ is independently H or D;

R² is H or D; and

R³ is CH₃ or CD₃;

provided that at least one of Y¹, Y², Y^{3a} and Y^{3b} is D.

In certain embodiments of the compositions of the invention, the compound of Formula Ia is a compound of Formula 3:

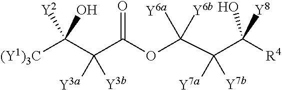

(3), or a pharmaceutically acceptable salt thereof, wherein:

each Y¹ is the same and is H or D;

each of Y², Y^{3a} and Y^{3b} is independently H or D;

each of Y^{6a}, Y^{6b}, Y^{7a}, Y^{7b} and Y⁸ is independently H or D; and

R⁴ is CH₃ or CD₃;

provided that at least one of Y¹, Y², Y^{3a} and Y^{3b} is D.

In certain embodiments of the compositions of the invention, the compound of Formula Ia is a compound of Formula 4:

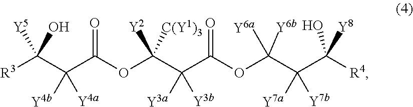

or a pharmaceutically acceptable salt thereof, wherein:

each Y¹ is the same and is H or D;

each of Y², Y^{3a} and Y^{3b} is independently H or D;

each of Y^{4a}, Y^{4b}, Y⁵, Y^{6a}, Y^{6b}, Y^{7a}, Y^{7b} and Y⁸ is independently H or D; and

R³ and R⁴ are independently CH₃ or CD₃;

provided that at least one of Y¹, Y², Y^{3a} and Y^{3b} is D.

In certain embodiments of the compositions of the invention, the compound is a compound of Formula Ib:

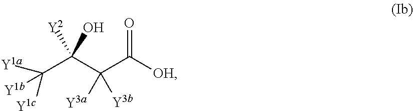

or a pharmaceutically acceptable salt thereof, wherein each of Y^{1a}, Y^{1b}, Y^{1c}, Y², Y^{3a} and Y^{3b} is independently H or D; provided that at least one of Y², Y^{3a} and Y^{3b} is D.

This invention also provides the use of compounds and compositions of the invention in methods of treating diseases and conditions that are beneficially treated by administering a BDNF. Some exemplary embodiments include a method of treating a disorder responsive to increased BDNF such as a neurological or neuropsychiatric condition including, but not limited to, Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, and eating disorders including anorexia nervosa and bulimia nervosa, the method comprising the step of administering to a subject in need thereof an effective amount of a compound or pharmaceutical composition of the present invention.

In certain embodiments, the method of treating diseases and conditions comprises the step of administering a compound of Formula I or a pharmaceutical composition comprising a compound of Formula I:

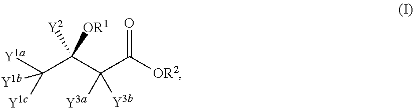

or a pharmaceutically acceptable salt thereof, wherein:

each of Y^{1a}, Y^{1b}, Y^{1c}, Y², Y^{3a} and Y^{3b} is independently H or D;

R¹ is H, D, —C(O)—C₁₋₆ alkyl, or

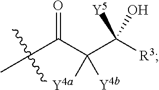

R² is H, D, —C₁₋₆ alkyl, —C₃₋₁₀ cycloalkyl, or

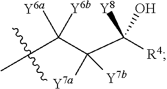

each of Y^{4a}, Y^{4b}, Y⁵, Y^{6a}, Y^{6b}, Y^{7a}, Y^{7b} and Y⁸, when present, is independently H or D; and

each of R³ and R⁴, when present, is CH₃ or CD₃;

provided that at least one of Y^{1a}, Y^{1b}, Y^{1c}, Y², Y^{3a} and Y^{3b} is D.

Further aspects and embodiments of the invention are also disclosed herein.

### BRIEF DESCRIPTION THE DRAWINGS

FIG. 1 is a graph showing the formation of NADH, resulting from the conversion of D-β-hydroxybutyric acid to acetyl acetate, as a function of incubation time in the enzymatic assay employing β-hydroxybutyrate dehydrogenase from Pseudomonas lemoignei at an initial concentration of 0.167 mM.

FIG. 2 is a substrate saturation plot generated from initial formation rate data in the enzymatic assay employing β-hydroxybutyrate dehydrogenase from Pseudomonas lemoignei and D-β-hydroxybutyric acid or a deuterated D-β-hydroxybutyric acid. Actual data points and Michaelis-Menten Model fitted curves are displayed.

### DETAILED DESCRIPTION OF THE INVENTION

DBHB, also known as D-β-hydroxybutyric acid and as D-3-hydroxybutyric acid, is a ketone body formed during exercise, or as a result of consuming a ketogenic diet. DBHB's inhibition of histone deacetylases HDAC2 and HDAC3 has been shown to increase levels of BDNF and thus to play a role in the positive effects of exercise on mood, memory and cognitive function that have been associated with BDNF.

BDNF has been associated with improvement in psychological states in normal humans, and its deficiency has been associated with numerous neurological and neuropsychiatric conditions, including Alzheimer's disease, Parkinson's disease, Huntington's disease, major depression, and post-traumatic stress disorder, among others. Both DBHB and direct administration of BDNF have been reported to show efficacy in preclinical models of Huntington's disease. See Lim et al. 2011, and Pollock et al., 2016. DBHB has been shown to protect neurons in models of Parkinson's Disease and Alzheimer's Disease. See Kashiwaya et al., 2000; Kashiwaya et al., 2012; and Tieu et al., 2003.

In humans, DBHB is oxidized by DBHB dehydrogenase to form acetoacetate, which enters the tricarboxylic acid cycle, rapidly forming glutamate, glutamine, and aspartate. Systemic DBHB is reported to cross the blood-brain barrier, resulting in increased central levels in humans. See Pan et al., J. Cereb. Blood Flow Metab., 2002 July; 22(7): 890-898, https://doi.org/10.1097/00004647-200207000-00014.

Despite the beneficial activities reported for BDNF and DBHB, there remains a need for improved treatments for neurological and neuropsychiatric diseases.

In one aspect, this invention relates to deuterated forms of DBHB, pharmaceutically acceptable salts thereof, analogs and prodrugs thereof, pharmaceutical compositions thereof, and methods of use.

### Definitions

The term “treat” means decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “subject” includes humans and non-human mammals. Non-limiting examples of non-human mammals include mice, rats, guinea pigs, rabbits, dogs, cats, monkeys, apes, pigs, cows, sheep, horses, etc. In certain embodiments, the subject is a human suffering from schizophrenia.

The term “alkyl” refers to a monovalent saturated hydrocarbon group. A C₁-C₄ alkyl is an alkyl having from 1 to 4 carbon atoms; a C₁-C₆ alkyl is an alkyl having from 1 to 6 carbon atoms. In some embodiments, an alkyl may be linear or branched. In some embodiments, an alkyl may be primary, secondary, or tertiary. Non-limiting examples of alkyl groups include methyl; ethyl; propyl, including n-propyl and isopropyl; butyl, including n-butyl, isobutyl, sec-butyl, and t-butyl; pentyl, including, for example, n-pentyl, isopentyl, and neopentyl; and hexyl, including, for example, n-hexyl and 2-methylpentyl. Non-limiting examples of primary alkyl groups include methyl, ethyl, n-propyl, n-butyl, n-pentyl, and n-hexyl. Non-limiting examples of secondary alkyl groups include isopropyl, sec-butyl, and 2-methylpentyl. Non-limiting examples of tertiary alkyl groups include t-butyl.

The term “cycloalkyl” refers to a monocyclic or bicyclic monovalent saturated or non-aromatic unsaturated hydrocarbon ring system. The term “C₃-C₁₀ cycloalkyl” refers to a cycloalkyl wherein the number of ring carbon atoms is from 3 to 10. Examples of C₃-C₁₀ cycloalkyl include C₃-C₆ cycloalkyl. Bicyclic ring systems include fused, bridged, and spirocyclic ring systems. More particular examples of cycloalkyl groups include, cyclopropyl, cyclobutyl, cyclopentyl, cyclohexyl, cycloheptyl, cis- and trans-decalinyl, norbornyl, and spiro[4.5]decanyl. In certain embodiments, the term “cycloalkyl” refers to a monocyclic or bicyclic monovalent saturated hydrocarbon ring system.

As used herein the term “pro-drug” refers to an agent which is converted into the parent drug in vivo by some physiological chemical process (e.g., a prodrug on being brought to the physiological pH is converted to the desired drug form). Pro-drugs are often useful because, in some situations, they may be easier to administer than the parent drug. They may, for instance, be bioavailable by oral administration whereas the parent drug is not. The pro-drug may also have improved solubility in pharmacological compositions over the parent drug. An example, without limitation, of a pro-drug would be a compound of the present invention wherein it is administered as an ester (the “pro-drug”) to facilitate transmittal across a cell membrane where water solubility is not beneficial, but then it is metabolically hydrolyzed to the carboxylic acid once inside the cell where water solubility is beneficial.

Pro-drugs have many useful properties. For example, a pro-drug may be more water soluble than the ultimate drug, thereby facilitating intravenous administration of the drug. A pro-drug may also have a higher level of oral bioavailability than the ultimate drug. After administration, the prodrug is enzymatically or chemically cleaved to deliver the ultimate drug in the blood or tissue.

Exemplary pro-drugs upon cleavage release the corresponding free acid, and such hydrolyzable ester-forming residues of the compounds of this invention include but are not limited to carboxylic acid substituents wherein the free hydrogen is replaced by (C₁-C₆)alkyl (such as methyl, ethyl, isopropyl, tert-butyl, neopentyl), (C₁-C₁₂)alkanoyloxymethyl, (C₄-C₉)1-(alkanoyloxy)ethyl, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di(C₁-C₂)-alkylcarbamoyl-(C₁-C₂)alkyl and piperidino-, pyrrolidino- or morpholino(C₂-C₃)alkyl.

Other exemplary pro-drugs release an alcohol of Formula (I) wherein the free hydrogen of the hydroxyl substituent (e.g., R¹) is replaced by (C₁-C₆)alkanoyloxymethyl, 1-((C₁-C₆)alkanoyloxy)ethyl, 1-methyl-1-((C₁-C₆)alkanoyloxy)ethyl, (C₁-C₁₂)alkoxycarbonyloxymethyl, N—(C₁-C₆)alkoxycarbonylamino-methyl, succinoyl, (C₁-C₆)alkanoyl, α-amino(C₁-C₄)alkanoyl, arylactyl and α-aminoacyl, or α-aminoacyl-α-aminoacyl wherein said α-aminoacyl moieties are independently any of the naturally occurring L-amino acids found in proteins, P(O)(OH)₂, —P(O)(O(C₁-C₆)alkyl)₂ or glycosyl (the radical resulting from detachment of the hydroxyl of the hemiacetal of a carbohydrate).

It will be recognized that some variation of natural isotopic abundance occurs in a synthesized compound depending upon the origin of chemical materials used in the synthesis. Thus, a preparation of Compound I will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada, E et al., Seikagaku, 1994, 66:15; Gannes, L Z et al., Comp Biochem Physiol Mol Integr Physiol, 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. However, in certain embodiments, when a position is designated specifically as “H” or “hydrogen”, the position has at least 80%, at least 90%, at least 95%, at least 96%, at least 97%, at least 98%, or at least 99% hydrogen. In some embodiments, when a position is designated specifically as “H” or “hydrogen”, the position incorporates ≤20% deuterium, ≤10% deuterium, ≤5% deuterium, ≤4% deuterium, ≤3% deuterium, ≤2% deuterium, or ≤1% deuterium. Also, unless otherwise stated, when a position is designated specifically as “D” or “deuterium”, the position is understood to have deuterium at an abundance that is at least 3340 times greater than the natural abundance of deuterium, which is 0.015% (i.e., at least 50.1% incorporation of deuterium).

The term “isotopic enrichment factor” as used herein means the ratio between the isotopic abundance and the natural abundance of a specified isotope.

In other embodiments, a compound of this invention has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, in a compound of this invention, each designated deuterium atom has deuterium incorporation of at least 52.5%. In some embodiments, in a compound of this invention, each designated deuterium atom has deuterium incorporation of at least 60%. In some embodiments, in a compound of this invention, each designated deuterium atom has deuterium incorporation of at least 67.5%. In some embodiments, in a compound of this invention, each designated deuterium atom has deuterium incorporation of at least 75%. In some embodiments, in a compound of this invention, each designated deuterium atom has deuterium incorporation of at least 82.5%. In some embodiments, in a compound of this invention, each designated deuterium atom has deuterium incorporation of at least 90%. In some embodiments, in a compound of this invention, each designated deuterium atom has deuterium incorporation of at least 95%. In some embodiments, in a compound of this invention, each designated deuterium atom has deuterium incorporation of at least 97%. In some embodiments, in a compound of this invention, each designated deuterium atom has deuterium incorporation of at least 97.5%. In some embodiments, in a compound of this invention, each designated deuterium atom has deuterium incorporation of at least 98%. In some embodiments, in a compound of this invention, each designated deuterium atom has deuterium incorporation of at least 99%. In some embodiments, in a compound of this invention, each designated deuterium atom has deuterium incorporation of at least 99.5%.

The term “isotopologue” refers to a molecule in which the chemical structure differs from another molecule of this invention only in the isotopic composition thereof.

The term “compound,” when referring to a compound of this invention, refers to a collection of molecules having an identical chemical structure, except that there may be isotopic variation among the constituent atoms of the molecules. Thus, it will be clear to those of skill in the art that a compound represented by a particular chemical structure will contain molecules having deuterium at each of the positions designated as deuterium in the chemical structure, and may also contain isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. In certain embodiments, the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

The invention also provides salts of the compounds of the invention.

A salt of a compound of this invention is formed between an acid and a basic group of the compound, such as an amino functional group, or a base and an acidic group of the compound, such as a carboxyl functional group. According to one embodiment, the compound is a pharmaceutically acceptable acid addition salt. In one embodiment the acid addition salt may be a deuterated acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

The pharmaceutically acceptable salt may be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

Certain compounds of the present invention (e.g., compounds of Formula I, I′, Ia, Ib, Ic, II, III, IIIa, 2, 3, 4, 5, 6 or 7) contain an asymmetric carbon atom (i.e., the carbon bearing the —OH group) and may contain one or more additional asymmetric carbon atoms. In certain embodiments, a compound of Formula I, I′, Ia, Ib, Ic, II, III, IIIa, 2, 3, 4, 5, 6 or 7 is substantially free from other possible stereoisomers, e.g., a compound of Formula I (or Ia) is substantially free of a compound of the structure:

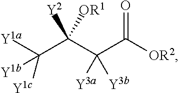

and a compound of Formula I (or Ia) is substantially free of a compound of the structure:

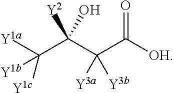

The term “substantially free of other stereoisomers” or “stereomerically pure” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

Unless otherwise indicated, when a disclosed compound is named or depicted by a structure having one or more chiral centers of unspecified stereochemistry, it is understood to represent all possible stereoisomers of the compound.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“Stereoisomer” refers to both enantiomers and diastereomers. “Tert” and “t-” each refer to tertiary. “Sec” or “s-” each refer to secondary. “n-” refers to normal. “i-” refers to iso. “US” refers to the United States of America.

“Substituted with deuterium” refers to the replacement of one or more hydrogen atoms with a corresponding number of deuterium atoms.

Throughout this specification, a variable may be referred to generally (e.g., “each Y¹”) or may be referred to specifically (e.g., Y^{1a}, Y^{1b}, Y^{1c}, etc.). Unless otherwise indicated, when a variable is referred to generally, it is meant to include all specific embodiments of that particular variable.

### Therapeutic Compounds and Compositions

In one aspect, the invention provides a compound of Formula I:

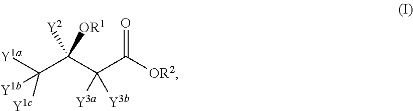

or a pharmaceutically acceptable salt thereof, wherein:

each of Y^{1a}, Y^{1b}, Y^{1c}, Y², Y^{3a} and Y^{3b} is independently H or D;

R¹ is H, D, —C(O)—C₁₋₆ alkyl, or

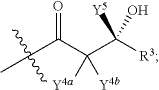

R² is H, D, —C₁₋₆ alkyl, —C₃₋₁₀ cycloalkyl,

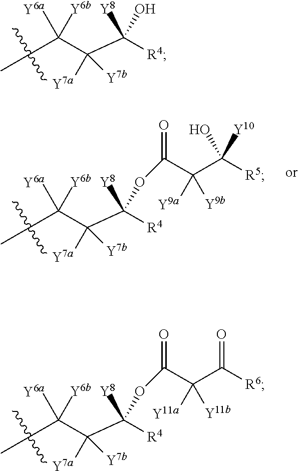

each of Y^{4a}, Y^{4b}, Y⁵, Y^{6a}, Y^{6b}, Y^{7a}, Y^{7b}, Y⁸, Y^{9a}, Y^{9b}, Y¹⁰, Y^{11a} and Y^{11b} when present, is independently H or D; and

each of R³, R⁴, R⁵ and R⁶, when present, is CH₃, CH₂D, CHD₂, or CD₃;

provided that at least one of Y^{1a}, Y^{1b}, Y^{1c}, Y², Y^{3a} and Y^{3b} is D.

In one embodiment of the compound of Formula I, the invention provides a compound of Formula I′:

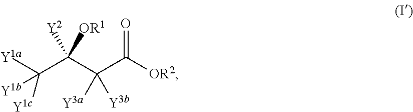

or a pharmaceutically acceptable salt thereof, wherein:

each of Y^{1a}, Y^{1b}, Y^{1c}, and Y² is independently H or D;

Y^{3a} and Y^{3b} are the same and are each H or each D;

R¹ is H, D, —C(O)—C₁₋₆ alkyl, or

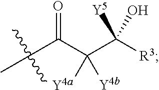

R² is H, D, —C₁₋₆ alkyl, —C₃₋₁₀ cycloalkyl,

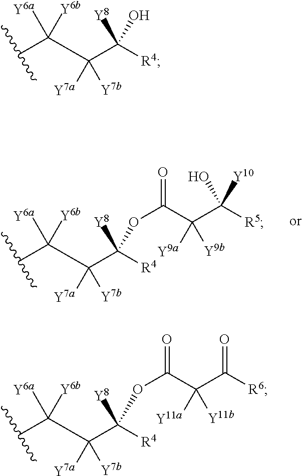

each of Y^{4a}, Y^{4b}, Y⁵, Y^{6a}, Y^{6b}, Y^{7a}, Y^{7b}, Y⁸, Y^{9a}, Y^{9b}, Y¹⁰, Y^{11a} and Y^{11b}, when present, is independently H or D; and

each of R³, R⁴, R⁵ and R⁶, when present, is CH₃, CH₂D, CHD₂, or CD₃;

provided that at least one of Y^{1a}, Y^{1b}, Y^{1c}, Y², Y^{3a} and Y^{3b} is D;
and further provided that the compound is not

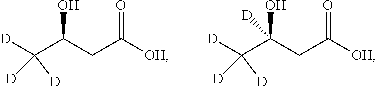 [SMILES: [2H]C([2H])([2H])[C@@H](O)CC(=O)O ; [2H]C([2H])([2H])[C@@]([2H])(O)CC(=O)O]

or a pharmaceutically acceptable salt thereof.

In another embodiment of the compound of Formula I, the invention provides a compound of Formula Ia:

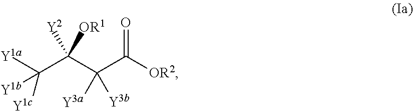

or a pharmaceutically acceptable salt thereof, wherein:

each of Y^{1a}, Y^{1b}, Y^{1c}, Y², Y^{3a} and Y^{3b} is independently H or D;

R¹ is H, D, —C(O)—C₁₋₆ alkyl, or

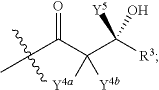

R² is H, D, —C₁₋₆ alkyl, —C₃₋₁₀ cycloalkyl,

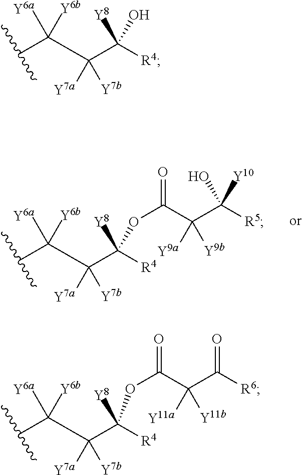

each of Y^{4a}, Y^{4b}, Y⁵, Y^{6a}, Y^{6b}, Y^{7a}, Y^{7b}, Y⁸, Y^{9a}, Y^{9b}, Y¹⁰, Y^{11a} and Y^{11b}, when present, is independently H or D;

each of R³, R⁴, R⁵ and R⁶, when present, is CH₃, CH₂D, CHD₂, or CD₃;

provided that at least one of Y^{1a}, Y^{1b}, Y^{1c}, Y², Y^{3a} and Y^{3b} is D;
and further provided that when R¹ is H, and R² is H, then at least one of Y², Y^{3a} and Y^{3b} is D.

In another embodiment of the compound of Formula I, the invention provides a compound of Formula Ia:

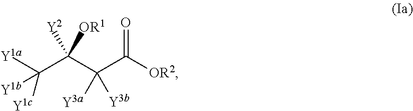

or a pharmaceutically acceptable salt thereof, wherein:

each of Y^{1a}, Y^{1b}, Y^{1c}, Y², Y^{3a} and Y^{3b} is independently H or D;

R¹ is H, D, —C(O)—C₁₋₆ alkyl, or

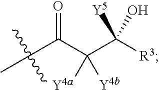

R² is H, D, —C₁₋₆ alkyl, —C₃₋₁₀ cycloalkyl,

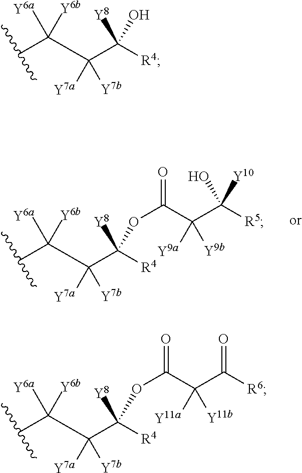

each of Y^{4a}, Y^{4b}, Y⁵, Y^{6a}, Y^{6b}, Y^{7a}, Y^{7b}, Y⁸, Y^{9a}, Y^{9b}, Y¹⁰, Y^{11a} and Y^{11b}, when present, is independently H or D;

each of R³, R⁴, R⁵ and R⁶, when present, is CH₃, CH₂D, CHD₂, or CD₃;

provided that at least one of Y², Y^{3a} and Y^{3b} is D.

In certain embodiments, the compound is a compound of Formula Ia, wherein

R² is H, D, —C₁₋₆ alkyl, —C₃₋₁₀ cycloalkyl, or

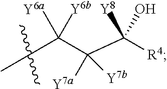

each of Y^{4a}, Y^{4b}, Y⁵, Y^{6a}, Y^{6b}, Y^{7a}, Y^{7b} and Y⁸, when present, is independently H or D; and

each of R³ and R⁴, when present, is independently CH₃ or CD₃.

In certain embodiments, the compound is a compound of Formula Ia, wherein

R¹ is

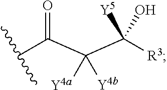

and wherein each of Y^{4a}, Y^{4b} and Y⁵ is independently H or D; and R³ is CH₃ or CD₃.

In certain embodiments, the compound is a compound of Formula Ia, wherein

R² is

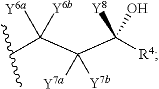

and wherein each of Y^{6a}, Y^{6b}, Y^{7a}, Y^{7b} and Y⁸ is independently H or D; and R⁴ is CH₃ or CD₃.

In certain embodiments of a compound of Formula Ia, each of Y^{1a}, Y^{1b}, and Y^{1c} is the same and the set of Y^{1a}, Y^{1b}, and Y^{1c} is represented as (Y¹)₃.

In certain embodiments of the compound of Formula I, I′ or Ia, R² is —C₃₋₆ cycloalkyl.

In certain embodiments, the compound of Formula Ia is a compound of Formula 2:

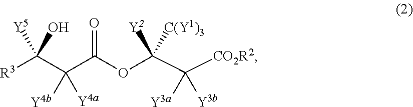

or a pharmaceutically acceptable salt thereof, wherein:

each Y¹ is the same and is H or D;

each of Y², Y^{3a} and Y^{3b} is independently H or D;

each of Y^{4a}, Y^{4b} and Y⁵ is independently H or D;

R² is H or D; and

R³ is CH₃ or CD₃;

provided that at least one of Y¹, Y², Y^{3a} and Y^{3b} is D.

In certain embodiments, the compound of Formula Ia is a compound of Formula 3:

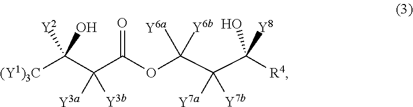

or a pharmaceutically acceptable salt thereof, wherein:

each Y¹ is the same and is H or D;

each of Y², Y^{3a} and Y^{3b} is independently H or D;

each of Y^{6a}, Y^{6b}, Y^{7a}, Y^{7b} and Y⁸ is independently H or D; and

R⁴ is CH₃ or CD₃;

provided that at least one of Y¹, Y², Y^{3a} and Y^{3b} is D.

In certain embodiments, the compound of Formula Ia is a compound of Formula 4:

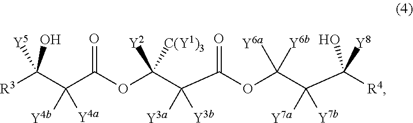

or a pharmaceutically acceptable salt thereof, wherein:

each Y¹ is the same and is H or D;

each of Y², Y^{3a} and Y^{3b} is independently H or D;

each of Y^{4a}, Y^{4b}, Y⁵, Y^{6a}, Y^{6b}, Y^{7a}, Y^{7b} and Y⁸ is independently H or D; and

R³ and R⁴ are independently CH₃ or CD₃;

provided that at least one of Y¹, Y², Y^{3a} and Y^{3b} is D.

In another embodiment of the compound of Formula I, the invention provides a compound of Formula Ib:

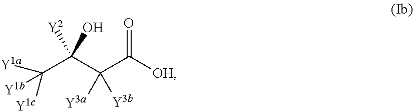

or a pharmaceutically acceptable salt thereof, wherein each of Y^{1a}, Y^{1b}, Y^{1c}, Y², Y^{3a} and Y^{3b} is independently H or D; provided that at least one of Y², Y^{3a} and Y^{3b} is D.

In another embodiment of the compound of Formula I, the invention provides a compound of Formula Ic:

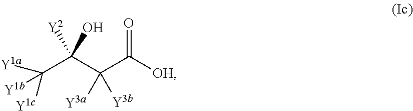

or a pharmaceutically acceptable salt thereof, wherein each of Y^{1a}, Y^{1b}, Y^{1c}, Y², Y^{3a} and Y^{3b} is independently H or D, provided that at least one of Y^{1a}, Y^{1b}, Y^{1c}, Y², Y^{3a} and Y^{3b} is D.

In another aspect, the invention provides a pharmaceutical composition comprising a compound of Formula I:

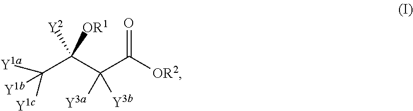

or a pharmaceutically acceptable salt thereof, wherein all variables are as defined for compounds of Formula I above; and a pharmaceutically acceptable carrier.

In another aspect, the invention provides a pharmaceutical composition comprising a compound of Formula I′:

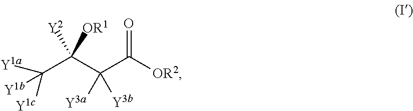

or a pharmaceutically acceptable salt thereof, wherein all variables are as defined for compounds of Formula I′ above; and a pharmaceutically acceptable carrier.

In certain aspects of the compositions of the invention, the compound is a compound of Formula Ia:

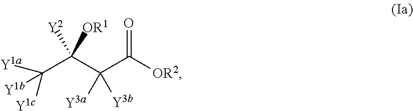

or a pharmaceutically acceptable salt thereof, wherein all variables are as defined for compounds of Formula Ia above.

In certain aspects of the compositions of the invention, the compound is a compound of Formula 2:

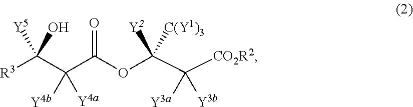

or a pharmaceutically acceptable salt thereof, wherein all variables are as defined for compounds of Formula 2 above.

In certain aspects of the compositions of the invention, the compound is a compound of Formula 3:

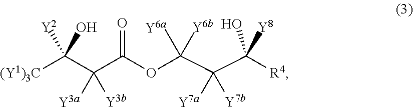

or a pharmaceutically acceptable salt thereof, wherein all variables are as defined for compounds of Formula 3 above.

In certain aspects of the compositions of the invention, the compound is a compound of Formula 4:

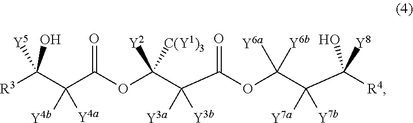

or a pharmaceutically acceptable salt thereof, wherein all variables are as defined for compounds of Formula 4 above.

In certain aspects of the compositions of the invention, the compound is a compound of Formula Ib:

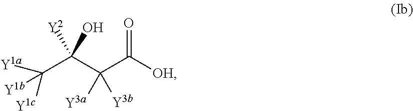

or a pharmaceutically acceptable salt thereof, wherein all variables are as defined for compounds of Formula Ib above.

In certain aspects of the compositions of the invention, the compound is a compound of Formula Ic:

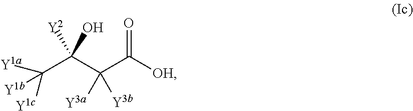

or a pharmaceutically acceptable salt thereof, wherein all variables are as defined for compounds of Formula Ic above.

In another aspect, the invention provides a compound of Formula II:

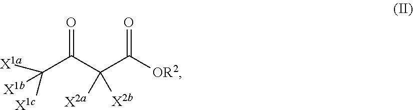

or a pharmaceutically acceptable salt thereof, wherein:

each of X^{1a}, X^{1b}, X^{1c}, X^{2a} and X^{2b} is independently H or D;

R² is

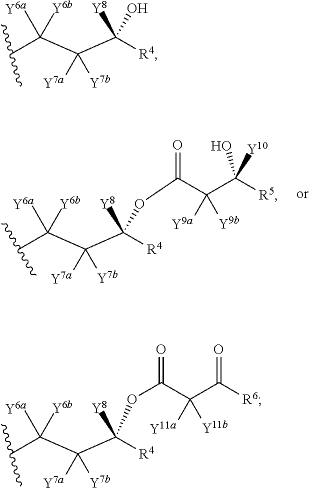

each of Y^{6a}, Y^{6b}, Y^{7a}, Y^{7b} and Y⁸ is independently H or D;

each of Y^{9a}, Y^{9b}, Y¹⁰, Y^{11a} and Y^{11b}, when present, is independently H or D;

R⁴ is CH₃, CH₂D, CHD₂, or CD₃;

each of R⁵ and R⁶, when present, is CH₃, CH₂D, CHD₂, or CD₃;

provided that at least one of X^{1a}, X^{1b}, X^{1c}, X^{2a} and X^{2b} is D.

In certain embodiments of a compound of Formula II, each of X^{1a}, X^{1b}, and X^{1c} is the same and the set of X^{1a}, X^{1b}, and X^{1c} is represented as (X¹)₃.

In one embodiment, the compound of Formula II is a compound of Formula 5:

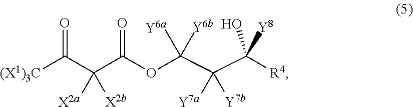

or a pharmaceutically acceptable salt thereof, wherein:

each X¹ is the same and is H or D;

each of X^{2a} and X^{2b} is independently H or D;

each of Y^{6a}, Y^{6b}, Y^{7a}, Y^{7b} and Y⁸ is independently H or D; and

R⁴ is CH₃, or CD₃;

provided that at least one of X¹, X^{2a} and X^{2b} is D.

In one embodiment, the compound of Formula II is a compound of Formula 6:

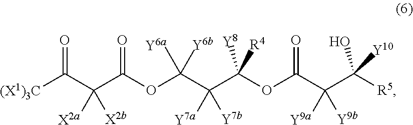

or a pharmaceutically acceptable salt thereof, wherein:

each X¹ is the same and is H or D;

each of X^{2a} and X^{2b} is independently H or D;

each of Y^{6a}, Y^{6b}, Y^{7a}, Y^{7b} and Y⁸ is independently H or D;

each of Y^{9a}, Y^{9b} and Y¹⁰ is independently H or D;

R⁴ is CH₃, or CD₃; and

R⁵ is CH₃, or CD₃;

provided that at least one of X¹, X^{2a} and X^{2b} is D.

In one embodiment, the compound of Formula II is a compound of Formula 7:

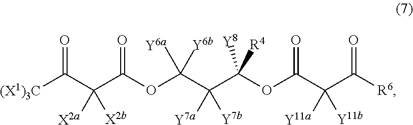

or a pharmaceutically acceptable salt thereof, wherein:

each X¹ is the same and is H or D;

each of X^{2a} and X^{2b} is independently H or D;

each of Y^{6a}, Y^{6b}, Y^{7a}, Y^{7b} and Y⁸ is independently H or D;

each of Y^{11a} and Y^{11b} is independently H or D;

R⁴ is CH₃ or CD₃; and

R⁶ is CH₃ or CD₃;

provided that at least one of X¹, X^{2a} and X^{2b} is D.

In another aspect, the invention provides a pharmaceutical composition comprising a compound of Formula II:

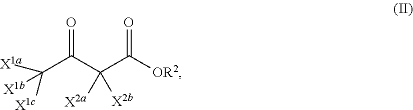

or a pharmaceutically acceptable salt thereof, wherein all variables are as defined for compounds of Formula II above; and a pharmaceutically acceptable carrier.

In certain aspects, the invention provides a pharmaceutical composition comprising a compound of Formula 5, or a pharmaceutically acceptable salt thereof, wherein all variables are as defined for compounds of Formula 5 above; and a pharmaceutically acceptable carrier.

In certain aspects, the invention provides a pharmaceutical composition comprising a compound of Formula 6, or a pharmaceutically acceptable salt thereof, wherein all variables are as defined for compounds of Formula 6 above; and a pharmaceutically acceptable carrier.

In certain aspects, the invention provides a pharmaceutical composition comprising a compound of Formula 7, or a pharmaceutically acceptable salt thereof, wherein all variables are as defined for compounds of Formula 7 above; and a pharmaceutically acceptable carrier.

In another aspect, the invention provides a compound of Formula III:

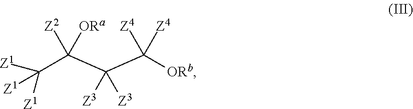

or a pharmaceutically acceptable salt thereof, wherein:

each Z¹ is the same and is H or D;

each Z² is H or D;

each Z³ is the same and is H or D;

each Z⁴ is the same and is H or D;

R^{a} is H or D; and

R^{b} is H or D;

provided that at least one of Z¹, Z², Z³ and Z⁴ is D;
and further provided that the compound is not

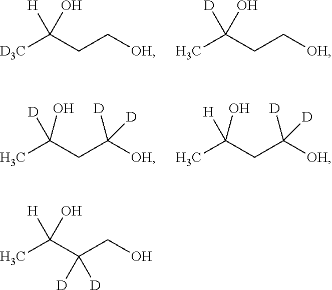

or a pharmaceutically acceptable salt thereof.

In another aspect, the invention provides a pharmaceutical composition comprising a compound of Formula III:

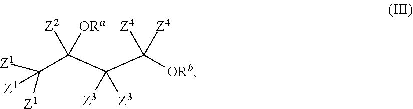

or a pharmaceutically acceptable salt thereof, wherein:

each Z¹ is the same and is H or D;

each Z² is H or D;

each Z³ is the same and is H or D;

each Z⁴ is the same and is H or D;

R^{a} is H or D; and

R^{b} is H or D;

provided that at least one of Z¹, Z², Z³ and Z⁴ is D;
or a pharmaceutically acceptable salt thereof, wherein all variables are as defined for compounds of Formula III above; and a pharmaceutically acceptable carrier.

In another aspect, the invention provides a compound of Formula IIIa:

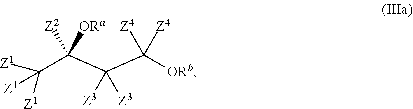

or a pharmaceutically acceptable salt thereof, wherein:

each Z¹ is the same and is H or D;

each Z² is H or D;

each Z³ is the same and is H or D;

each Z⁴ is the same and is H or D;

R^{a} is H or D; and

R^{b} is H or D;

provided that at least one of Z¹, Z², Z³ and Z⁴ is D;
or a pharmaceutically acceptable salt thereof.

In certain embodiments of a compound of Formula III or IIIa, each Z¹ is D and Z² is D. In other embodiments of a compound of Formula III or IIIa, each Z¹ is D and each Z³ is D. In other embodiments of a compound of Formula III or IIIa, each Z¹ is D and each Z⁴ is D.

In certain embodiments of a compound of Formula III or IIIa, Z² is D and each Z³ is D. In other embodiments of a compound of Formula III or IIIa, Z² is D and each Z⁴ is D.

In certain embodiments of a compound of Formula III or IIIa, each Z³ is D and each Z⁴ is D.

In certain embodiments of a compound of Formula IIIa, the compound is not

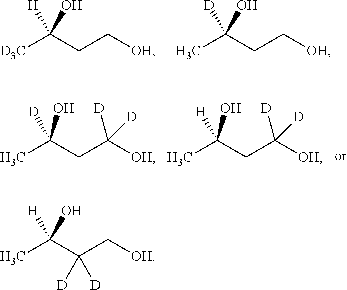

In another aspect, the invention provides a pharmaceutical composition comprising a compound of Formula IIIa:

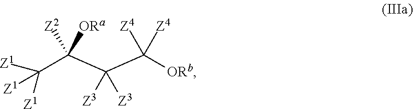

or a pharmaceutically acceptable salt thereof, wherein:

each Z¹ is the same and is H or D;

each Z² is H or D;

each Z³ is the same and is H or D;

each Z⁴ is the same and is H or D;

R^{a} is H or D; and

R^{b} is H or D;

provided that at least one of Z¹, Z², Z³ and Z⁴ is D;
or a pharmaceutically acceptable salt thereof, wherein all variables are as defined for compounds of Formula IIIa above; and a pharmaceutically acceptable carrier.

In certain aspects of the compounds of Formula I, I′, Ia, Ib or Ic, Y^{1a}, Y^{1b} and Y^{1c} are the same and are each H or each D. In one embodiment Y^{1a}, Y^{1b} and Y^{1c} are each D. In an alternate embodiment Y^{1a}, Y^{1b} and Y^{1c} are each H.

In certain aspects of the compounds of Formula I, I′, Ia, Ib or Ic, Y^{3a} and Y^{3b} are the same and are each H or each D. In one embodiment, Y^{3a} and Y^{3b} are each D. In an alternate embodiment, Y^{3a} and Y^{3b} are each H.

In certain aspects of the compounds of Formula 2, 3 or 4, Y^{3a} and Y^{3b} are the same and are each H or each D. In one embodiment, Y^{3a} and Y^{3b} are each D. In an alternate embodiment, Y^{3a} and Y^{3b} are each H.

In certain embodiments of the compounds of Formula I, I′, Ia, Ib or Ic, Y² is D.

In certain embodiments of the compounds of Formula I, I′, Ia, Ib or Ic, Y² is H.

In certain embodiments of the compounds of Formula 2, 3 or 4, Y² is D.

In certain embodiments of the compounds of Formula 2, 3 or 4, Y² is H.

In certain aspects of the compounds of Formula II, X^{1a}, X^{1b} and X^{1c} are the same and are each H or each D. In one embodiment, X^{1a}, X^{1b} and X^{1c} are each D. In an alternate embodiment, X^{1a}, X^{1b} and X^{1c} are each H.

In certain aspects of the compounds of Formula II, X^{2a} and X^{2b} are the same and are each H or each D. In one embodiment, X^{2a} and X^{2b} are each D. In an alternate embodiment, X^{2a} and X^{2b} are each H.

In certain aspects of the compounds of Formula 5, 6, or 7, X^{2a} and X^{2b} are the same and are each H or each D. In one embodiment, X^{2a} and X^{2b} are each D. In an alternate embodiment, X^{2a} and X^{2b} are each H.

In certain embodiments of the compounds of Formula I, I′, Ia, Ib, Ic or II, each position designated specifically as deuterium has at least 90% incorporation of deuterium.

In certain embodiments of the compounds of Formula I, I′, Ia, Ib, Ic or II, each position designated specifically as deuterium has at least 95% incorporation of deuterium.

In certain embodiments of the compounds of Formula 2, 3, 4, 5, 6 or 7, each position designated specifically as deuterium has at least 90% incorporation of deuterium.

In certain embodiments of the compounds of Formula 2, 3, 4, 5, 6 or 7, each position designated specifically as deuterium has at least 95% incorporation of deuterium.

In certain embodiments of the compounds of Formula III or IIIa, each position designated specifically as deuterium has at least 90% incorporation of deuterium.

In certain embodiments of the compounds of Formula III or IIIa, each position designated specifically as deuterium has at least 95% incorporation of deuterium.

In certain embodiments of any of the compounds of this invention, each position designated specifically as deuterium has at least 98% incorporation of deuterium.

In some embodiments of a compound of this invention, when Y^{1a}, Y^{1b} or Y^{1c} is deuterium, the level of deuterium incorporation at each Y^{1a}, Y^{1b} or Y^{1c} designated as deuterium is at least 52.5%, at least 75%, at least 82.5%, at least 90%, at least 95%, at least 97%, or at least 99%.

In some embodiments of a compound of this invention, when Y^{1a}, Y^{1b} and Y^{1c} are deuterium, the level of deuterium incorporation at each of Y^{1a}, Y^{1b} and Y^{1c} is at least 52.5%, at least 75%, at least 82.5%, at least 90%, at least 95%, at least 97%, or at least 99%.

In some embodiments of a compound of this invention, when Y² is deuterium, the level of deuterium incorporation at Y² is at least 52.5%, at least 75%, at least 82.5%, at least 90%, at least 95%, at least 97%, or at least 99%.

In some embodiments of a compound of this invention, when Y^{3a} or Y^{3b} is deuterium, the level of deuterium incorporation at each Y^{3a} or Y^{3b} designated as deuterium is at least 52.5%, at least 75%, at least 82.5%, at least 90%, at least 95%, at least 97%, or at least 99%.

In some embodiments of a compound of this invention, when Y^{3a} and Y^{3b} are deuterium, the level of deuterium incorporation at each of Y^{3a} and Y^{3b} is at least 52.5%, at least 75%, at least 82.5%, at least 90%, at least 95%, at least 97%, or at least 99%.

In some embodiments of a compound of this invention, when Y^{4a} or Y^{4b} is deuterium, the level of deuterium incorporation at each Y^{4a} or Y^{4b} designated as deuterium is at least 52.5%, at least 75%, at least 82.5%, at least 90%, at least 95%, at least 97%, or at least 99%.

In some embodiments of a compound of this invention, when Y^{4a} and Y^{4b} are deuterium, the level of deuterium incorporation at each of Y^{4a} and Y^{4b} is at least 52.5%, at least 75%, at least 82.5%, at least 90%, at least 95%, at least 97%, or at least 99%.

In some embodiments of a compound of this invention, when Y⁵ is deuterium, the level of deuterium incorporation at Y⁵ is at least 52.5%, at least 75%, at least 82.5%, at least 90%, at least 95%, at least 97%, or at least 99%.

In some embodiments of a compound of this invention, when Y^{6a} or Y^{6b} is deuterium, the level of deuterium incorporation at each Y^{6a} or Y^{6b} designated as deuterium is at least 52.5%, at least 75%, at least 82.5%, at least 90%, at least 95%, at least 97%, or at least 99%.

In some embodiments of a compound of this invention, when Y^{6a} and Y^{6b} are deuterium, the level of deuterium incorporation at each of Y^{6a} and Y^{6b} designated as deuterium is at least 52.5%, at least 75%, at least 82.5%, at least 90%, at least 95%, at least 97%, or at least 99%.

In some embodiments of a compound of this invention, when Y^{7a} or Y^{7b} is deuterium, the level of deuterium incorporation at each Y^{7a} or Y^{7b} designated as deuterium is at least 52.5%, at least 75%, at least 82.5%, at least 90%, at least 95%, at least 97%, or at least 99%.

In some embodiments of a compound of this invention, when Y^{7a} and Y^{7b} are deuterium, the level of deuterium incorporation at each of Y^{7a} and Y^{7b} is at least 52.5%, at least 75%, at least 82.5%, at least 90%, at least 95%, at least 97%, or at least 99%.

In some embodiments of a compound of this invention, when Y⁸ is deuterium, the level of deuterium incorporation at Y⁸ is at least 52.5%, at least 75%, at least 82.5%, at least 90%, at least 95%, at least 97%, or at least 99%.

In some embodiments of a compound of this invention, when Y^{9a} or Y^{9b} is deuterium, the level of deuterium incorporation at each Y^{9a} or Y^{9b} designated as deuterium is at least 52.5%, at least 75%, at least 82.5%, at least 90%, at least 95%, at least 97%, or at least 99%.

In some embodiments of a compound of this invention, when Y^{9a} and Y^{9b} are deuterium, the level of deuterium incorporation at each of Y^{9a} and Y^{9b} is at least 52.5%, at least 75%, at least 82.5%, at least 90%, at least 95%, at least 97%, or at least 99%.

In some embodiments of a compound of this invention, when Y¹⁰ is deuterium, the level of deuterium incorporation at Y¹⁰ is at least 52.5%, at least 75%, at least 82.5%, at least 90%, at least 95%, at least 97%, or at least 99%.

In some embodiments of a compound of this invention, when Y^{11a} or Y^{11b} is deuterium, the level of deuterium incorporation at each Y^{11a} or Y^{11b} designated as deuterium is at least 52.5%, at least 75%, at least 82.5%, at least 90%, at least 95%, at least 97%, or at least 99%.

In some embodiments of a compound of this invention, when Y^{11a} and Y^{11b} are deuterium, the level of deuterium incorporation at each of Y^{11a} and Y^{11b} is at least 52.5%, at least 75%, at least 82.5%, at least 90%, at least 95%, at least 97%, or at least 99%.

In some embodiments of a compound of this invention, when X^{1a}, X^{1b} or X^{1c} is deuterium, the level of deuterium incorporation at each X^{1a}, X^{1b} or X^{1c} designated as deuterium is at least 52.5%, at least 75%, at least 82.5%, at least 90%, at least 95%, at least 97%, or at least 99%.

In some embodiments of a compound of this invention, when X^{1a}, X^{1b} or X^{1c} are deuterium, the level of deuterium incorporation at each of X^{1a}, X^{1b} or X^{1c} is at least 52.5%, at least 75%, at least 82.5%, at least 90%, at least 95%, at least 97%, or at least 99%.

In some embodiments of a compound of this invention, when X^{2a} or X^{2b} is deuterium, the level of deuterium incorporation at each X^{2a} or X^{2b} designated as deuterium is at least 52.5%, at least 75%, at least 82.5%, at least 90%, at least 95%, at least 97%, or at least 99%.

In some embodiments of a compound of this invention, when Z¹ is deuterium, the level of deuterium incorporation at each Z¹ designated as deuterium is at least 52.5%, at least 75%, at least 82.5%, at least 90%, at least 95%, at least 97%, or at least 99%.

In some embodiments of a compound of this invention, when Z² is deuterium, the level of deuterium incorporation at each Z² designated as deuterium is at least 52.5%, at least 75%, at least 82.5%, at least 90%, at least 95%, at least 97%, or at least 99%.

In some embodiments of a compound of this invention, when Z³ is deuterium, the level of deuterium incorporation at each Z³ designated as deuterium is at least 52.5%, at least 75%, at least 82.5%, at least 90%, at least 95%, at least 97%, or at least 99%.

In some embodiments of a compound of this invention, when Z⁴ is deuterium, the level of deuterium incorporation at each Z⁴ designated as deuterium is at least 52.5%, at least 75%, at least 82.5%, at least 90%, at least 95%, at least 97%, or at least 99%.

In some embodiments of a compound of this invention, when R^{a} is deuterium, the level of deuterium incorporation at each R^{a} designated as deuterium is at least 52.5%, at least 75%, at least 82.5%, at least 90%, at least 95%, at least 97%, or at least 99%.

In some embodiments of a compound of this invention, when R^{b} is deuterium, the level of deuterium incorporation at each R^{b} designated as deuterium is at least 52.5%, at least 75%, at least 82.5%, at least 90%, at least 95%, at least 97%, or at least 99%.

In certain embodiments, in the compound of Formula I, I′, Ia, Ib, Ic, II, III, or IIIa, any atom not designated as deuterium is present at its natural isotopic abundance.

In some embodiments of a compound of this invention, at least one of Y^{1a}, Y^{1b}, Y^{1c}, Y², Y^{3a} and Y^{3b}, is hydrogen.

In some embodiments of a compound of this invention, at least one of X^{1a}, X^{1b}, X^{1c}, X^{2a} and X^{2b}, is hydrogen.

In some embodiments of a compound of Formula III or IIIa, at least one of Z¹, Z², Z³ and Z⁴, is hydrogen.

In certain embodiments, the compound of Formula I, I′, Ia, Ib, Ic or II is at least about 90% stereomerically pure, e.g., for a compound of Formula I, the compound comprises at least 90% of the structure

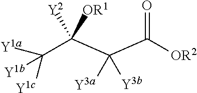

and not more than 10% of

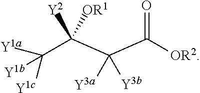

In certain embodiments, the compound of Formula IIIa is at least about 90% stereomerically pure, e.g., for a compound of Formula IIIa, the compound comprises at least 90% of the structure

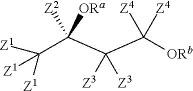

and not more than 10% of

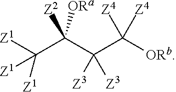

A compound of Formula I, I′, Ia, Ib, Ic, or II may exist as a zwitterion (e.g., a compound of Formula I can be represented by the structure:

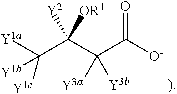

It will be understood that such zwitterionic forms are included within the scope of this invention.

In certain embodiments, the pharmaceutical composition is suitable for oral administration.

In certain embodiments, the pharmaceutical composition comprises 0.1 g to 60 g of the compound of Formula I, I′, Ia, Ib, Ic, or II. In certain aspects the pharmaceutical composition comprises 5 g to 30 g of the compound of Formula I, I′, Ia, Ib, Ic, or II. In certain aspects the pharmaceutical composition comprises 10 g to 20 g of the compound of Formula I, I′, Ia, Ib, Ic, or II. In certain aspects the pharmaceutical composition comprises 0.5 g to 10 g of the compound of Formula I, I′, Ia, Ib, Ic, or II. In certain aspects the pharmaceutical composition comprises 0.5 g to 3 g of the compound of Formula I, I′, Ia, Ib, Ic, or II.

In some embodiments, the compound is a compound of Formula Ib, wherein Y^{1a}, Y^{1b} and Y^{1c} are the same; Y² is D; and the compound is selected from any one of the compounds set forth in Table 1a (below):

TABLE 1a
Exemplary Embodiments of Formula Ib

| | Each Y^{1a}, | | |
|---|---|---|---|
| Compound # | Y^{1b} and Y^{1c} | Y^{3a} | Y^{3b} |
| 100 | H | H | H |
| 101 | H | H | D |
| 102 | D | H | H |
| 103 | D | D | H |
| 104 | H | D | D |
| 105 | D | D | D |

or a pharmaceutically acceptable salt thereof.

In some embodiments, the compound is a compound of Formula Ib, wherein Y^{1a}, Y^{1b} and Y^{1c} are the same; Y² is D; and the compound is selected from any one of the compounds set forth in Table 1aa (below):

TABLE 1aa
Exemplary Embodiments of Formula Ib

| | Each Y^{1a}, | | |
|---|---|---|---|
| Compound # | Y^{1b} and Y^{1c} | Y^{3a} | Y^{3b} |
| 100 | H | H | H |
| 101 | H | H | D |
| 103 | D | D | H |
| 104 | H | D | D |
| 105 | D | D | D |

or a pharmaceutically acceptable salt thereof.

In some embodiments, the compound is a compound of Formula Ib or Ic, wherein Y^{1a}, Y^{1b} and Y^{1c} are the same, Y² is H, and the compound is selected from any one of the compounds set forth in Table 1b (below):

TABLE 1b
Exemplary Embodiments of Formula Ib and Ic

| | Each Y^{1a}, | | |
|---|---|---|---|
| Compound # | Y^{1b} and Y^{1c} | Y^{3a} | Y^{3b} |
| 201 | H | H | D |
| 202 | D | H | H |
| 203 | D | D | H |
| 204 | H | D | D |
| 205 | D | D | D |

or a pharmaceutically acceptable salt thereof.

In some embodiments, the compound is a compound of Formula Ib or Ic, wherein Y^{1a}, Y^{1b} and Y^{1c} are the same, Y² is H, and the compound is selected from any one of the compounds set forth in Table 1bb (below):

TABLE 1bb
Exemplary Embodiments of Formula Ib and Ic

| | Each Y^{1a}, | | |
|---|---|---|---|
| Compound # | Y^{1b} and Y^{1c} | Y^{3a} | Y^{3b} |
| 201 | H | H | D |
| 203 | D | D | H |
| 204 | H | D | D |
| 205 | D | D | D |

or a pharmaceutically acceptable salt thereof.

In some embodiments, the compound is a compound of Formula 2, wherein each Y¹ is the same; Y² is D; Y^{3a} and Y^{3b} are the same; Y^{4a} and Y^{4b} are the same; R² is H; and the compound is selected from any one of the compounds set forth in Table 2a (below):

TABLE 2a
Exemplary Embodiments of Formula 2

| Compound # | Each Y¹ | Each Y^{3a}/Y^{3b} | Each Y^{4a}/Y^{4b} | Y⁵ | R³ |
|---|---|---|---|---|---|
| 300 | H | H | H | H | CH₃ |
| 301 | D | H | H | H | CH₃ |
| 302 | H | D | H | H | CH₃ |
| 303 | H | H | D | H | CH₃ |
| 304 | H | H | H | D | CH₃ |
| 305 | H | H | H | H | CD₃ |
| 306 | D | D | H | H | CH₃ |
| 307 | D | H | D | H | CH₃ |
| 308 | D | H | H | D | CH₃ |
| 309 | D | H | H | H | CD₃ |
| 310 | H | D | D | H | CH₃ |
| 311 | H | D | H | D | CH₃ |
| 312 | H | D | H | H | CD₃ |
| 313 | H | H | D | D | CH₃ |
| 314 | H | H | D | H | CD₃ |
| 315 | H | H | H | D | CD₃ |
| 316 | D | D | D | H | CH₃ |
| 317 | D | D | H | D | CH₃ |
| 318 | D | D | H | H | CD₃ |
| 319 | D | H | D | D | CH₃ |
| 320 | D | H | D | H | CD₃ |
| 321 | D | H | H | D | CD₃ |
| 322 | H | D | D | D | CH₃ |
| 323 | H | D | D | H | CD₃ |
| 324 | H | D | H | D | CD₃ |
| 325 | H | H | D | D | CD₃ |
| 326 | D | D | D | D | CH₃ |
| 327 | D | D | D | H | CD₃ |
| 328 | D | D | H | D | CD₃ |
| 329 | D | H | D | D | CD₃ |
| 330 | H | D | D | D | CD₃ |
| 331 | D | D | D | D | CD₃ |

or a pharmaceutically acceptable salt thereof.

In some embodiments, the compound is a compound of Formula 2, wherein each Y¹ is the same; Y² is H; Y^{3a} and Y^{3b} are the same; Y^{4a} and Y^{4b} are the same; R² is H; and the compound is selected from any one of the compounds set forth in Table 2b (below):

TABLE 2b
Exemplary Embodiments of Formula 2

| Compound # | Each Y¹ | Each Y^{3a}/Y^{3b} | Each Y^{4a}/Y^{4b} | Y⁵ | R³ |
|---|---|---|---|---|---|
| 401 | D | H | H | H | CH₃ |
| 402 | H | D | H | H | CH₃ |
| 403 | H | H | D | H | CH₃ |
| 404 | H | H | H | D | CH₃ |
| 405 | H | H | H | H | CD₃ |
| 406 | D | D | H | H | CH₃ |
| 407 | D | H | D | H | CH₃ |
| 408 | D | H | H | D | CH₃ |
| 409 | D | H | H | H | CD₃ |
| 410 | H | D | D | H | CH₃ |
| 411 | H | D | H | D | CH₃ |
| 412 | H | D | H | H | CD₃ |
| 413 | H | H | D | D | CH₃ |
| 414 | H | H | D | H | CD₃ |
| 415 | H | H | H | D | CD₃ |
| 416 | D | D | D | H | CH₃ |
| 417 | D | D | H | D | CH₃ |
| 418 | D | D | H | H | CD₃ |
| 419 | D | H | D | D | CH₃ |
| 420 | D | H | D | H | CD₃ |
| 421 | D | H | H | D | CD₃ |
| 422 | H | D | D | D | CH₃ |
| 423 | H | D | D | H | CD₃ |
| 424 | H | D | H | D | CD₃ |
| 425 | H | H | D | D | CD₃ |
| 426 | D | D | D | D | CH₃ |
| 427 | D | D | D | H | CD₃ |
| 428 | D | D | H | D | CD₃ |
| 429 | D | H | D | D | CD₃ |
| 430 | H | D | D | D | CD₃ |
| 431 | D | D | D | D | CD₃ |

or a pharmaceutically acceptable salt thereof.

In some embodiments, the compound is a compound of Formula 3, wherein each Y¹ is the same; Y² is D; Y^{3a} and Y^{3b} are the same; Y^{6a}, Y^{6b}, Y^{7a} and Y^{7b} are the same; and the compound is selected from any one of the compounds set forth in Table 3a (below):

TABLE 3a
Exemplary Embodiments of Formula 3

| | | Each Y^{3a}/ | Each Y^{6a}/Y^{6b} | | |
|---|---|---|---|---|---|
| Compound # | Each Y¹ | Y^{3b} | Y^{7a}/Y^{7b} | Y⁸ | R⁴ |
| 500 | H | H | H | H | CH₃ |
| 501 | D | H | H | H | CH₃ |
| 502 | H | D | H | H | CH₃ |
| 503 | H | H | D | H | CH₃ |
| 504 | H | H | H | D | CH₃ |
| 505 | H | H | H | H | CD₃ |
| 506 | D | D | H | H | CH₃ |
| 507 | D | H | D | H | CH₃ |
| 508 | D | H | H | D | CH₃ |
| 509 | D | H | H | H | CD₃ |
| 510 | H | D | D | H | CH₃ |
| 511 | H | D | H | D | CH₃ |
| 512 | H | D | H | H | CD₃ |
| 513 | H | H | D | D | CH₃ |
| 514 | H | H | D | H | CD₃ |
| 515 | H | H | H | D | CD₃ |
| 516 | D | D | D | H | CH₃ |
| 517 | D | D | H | D | CH₃ |
| 518 | D | D | H | H | CD₃ |
| 519 | D | H | D | D | CH₃ |
| 520 | D | H | D | H | CD₃ |
| 521 | D | H | H | D | CD₃ |
| 522 | H | D | D | D | CH₃ |
| 523 | H | D | D | H | CD₃ |
| 524 | H | D | H | D | CD₃ |
| 525 | H | H | D | D | CD₃ |
| 526 | D | D | D | D | CH₃ |
| 527 | D | D | D | H | CD₃ |
| 528 | D | D | H | D | CD₃ |
| 529 | D | H | D | D | CD₃ |
| 530 | H | D | D | D | CD₃ |
| 531 | D | D | D | D | CD₃ |

or a pharmaceutically acceptable salt thereof.

In some embodiments, the compound is a compound of Formula 3, wherein each Y¹ is the same; Y² is H; Y^{3a} and Y^{3b} are the same; Y^{6a}, Y^{6b}, Y^{7a} and Y^{7b} are the same; and the compound is selected from any one of the compounds set forth in Table 3b (below):

TABLE 3b
Exemplary Embodiments of Formula 3

| | | | Each Y^{6a}/Y^{6b} | | |
|---|---|---|---|---|---|
| Compound # | Each Y¹ | Each Y^{3a}/Y^{3b} | Y^{7a}/Y^{7b} | Y⁸ | R⁴ |
| 601 | D | H | H | H | CH₃ |
| 602 | H | D | H | H | CH₃ |
| 603 | H | H | D | H | CH₃ |
| 604 | H | H | H | D | CH₃ |
| 605 | H | H | H | H | CD₃ |
| 606 | D | D | H | H | CH₃ |
| 607 | D | H | D | H | CH₃ |
| 608 | D | H | H | D | CH₃ |
| 609 | D | H | H | H | CD₃ |
| 610 | H | D | D | H | CH₃ |
| 611 | H | D | H | D | CH₃ |
| 612 | H | D | H | H | CD₃ |
| 613 | H | H | D | D | CH₃ |
| 614 | H | H | D | H | CD₃ |
| 615 | H | H | H | D | CD₃ |
| 616 | D | D | D | H | CH₃ |
| 617 | D | D | H | D | CH₃ |
| 618 | D | D | H | H | CD₃ |
| 619 | D | H | D | D | CH₃ |
| 620 | D | H | D | H | CD₃ |
| 621 | D | H | H | D | CD₃ |
| 622 | H | D | D | D | CH₃ |
| 623 | H | D | D | H | CD₃ |
| 624 | H | D | H | D | CD₃ |
| 625 | H | H | D | D | CD₃ |
| 626 | D | D | D | D | CH₃ |
| 627 | D | D | D | H | CD₃ |
| 628 | D | D | H | D | CD₃ |
| 629 | D | H | D | D | CD₃ |
| 630 | H | D | D | D | CD₃ |
| 631 | D | D | D | D | CD₃ |

or a pharmaceutically acceptable salt thereof.

In some embodiments, the compound is a compound of Formula 4, wherein Y² is D; Y^{3a} and Y^{3b} are the same; Y^{4a} and Y^{4b} are the same; Y⁵ is D; Y^{6a} and Y^{6b} are each H; Y^{7a} and Y^{7b} are the same; Y⁸ is D; R³ and R⁴ are the same; and the compound is selected from any one of the compounds set forth in Table 4a (below):

TABLE 4a
Exemplary Embodiments of Formula 4

| | | Each Y^{3a}/ | Each Y^{4a}/ | Each Y^{7a}/ | |
|---|---|---|---|---|---|
| Compound # | Each Y¹ | Y^{3b} | Y^{4b} | Y^{7b} | R³/R⁴ |
| 650a | H | H | H | H | CH₃ |
| 651a | D | H | H | H | CH₃ |
| 652a | H | D | H | H | CH₃ |
| 653a | H | H | D | H | CH₃ |
| 654a | H | H | H | D | CH₃ |
| 655a | H | H | H | H | CD₃ |
| 656a | D | D | H | H | CH₃ |
| 657a | D | H | D | H | CH₃ |
| 658a | D | H | H | D | CH₃ |
| 659a | D | H | H | H | CD₃ |
| 660a | H | D | D | H | CH₃ |
| 661a | H | D | H | D | CH₃ |
| 662a | H | D | H | H | CD₃ |
| 663a | H | H | D | D | CH₃ |
| 664a | H | H | D | H | CD₃ |
| 665a | H | H | H | D | CD₃ |
| 666a | D | D | D | H | CH₃ |
| 667a | D | D | H | D | CH₃ |
| 668a | D | D | H | H | CD₃ |
| 669a | D | H | D | D | CH₃ |
| 670a | D | H | D | H | CD₃ |
| 671a | D | H | H | D | CD₃ |
| 672a | H | D | D | D | CH₃ |
| 673a | H | D | D | H | CD₃ |
| 674a | H | D | H | D | CD₃ |
| 675a | H | H | D | D | CD₃ |
| 676a | D | D | D | D | CH₃ |
| 677a | D | D | D | H | CD₃ |
| 678a | D | D | H | D | CD₃ |
| 679a | D | H | D | D | CD₃ |
| 680a | H | D | D | D | CD₃ |
| 681a | D | D | D | D | CD₃ |

or a pharmaceutically acceptable salt thereof.

In some embodiments, the compound is a compound of Formula 4, wherein Y² is D; Y^{3a} and Y^{3b} are the same; Y^{4a} and Y^{4b} are the same; Y⁵ is D; Y^{6a} and Y^{6b} are each H; Y^{7a} and Y^{7b} are the same; Y⁸ is D; R³ is CD₃ and R⁴ is CH₃; and the compound is selected from any one of the compounds set forth in Table 4b (below):

TABLE 4b
Exemplary Embodiments of Formula 4

| | | Each Y^{3a}/ | Each Y^{4a}/ | Each Y^{7a}/ |
|---|---|---|---|---|
| Compound # | Each Y¹ | Y^{3b} | Y^{4b} | Y^{7b} |
| 650b | H | H | H | H |
| 651b | D | H | H | H |
| 652b | H | D | H | H |
| 653b | H | H | D | H |
| 654b | H | H | H | D |
| 656b | D | D | H | H |
| 657b | D | H | D | H |
| 658b | D | H | H | D |
| 660b | H | D | D | H |
| 661b | H | D | H | D |
| 663b | H | H | D | D |
| 666b | D | D | D | H |
| 667b | D | D | H | D |
| 669b | D | H | D | D |
| 672b | H | D | D | D |
| 676b | D | D | D | D |

or a pharmaceutically acceptable salt thereof.

In some embodiments, the compound is a compound of Formula 4, wherein Y² is D; Y^{3a} and Y^{3b} are the same; Y^{4a} and Y^{4b} are the same; Y⁵ is D; Y^{6a} and Y^{6b} are each H; Y^{7a} and Y^{7b} are the same; Y⁸ is D; R³ is CH₃ and R⁴ is CD₃; and the compound is selected from any one of the compounds set forth in Table 4c (below):

TABLE 4c
Exemplary Embodiments of Formula 4

| | | Each Y^{3a}/ | Each Y^{4a}/ | Each Y^{7a}/ |
|---|---|---|---|---|
| Compound # | Each Y¹ | Y^{3b} | Y^{4b} | Y^{7b} |
| 650c | H | H | H | H |
| 651c | D | H | H | H |
| 652c | H | D | H | H |
| 653c | H | H | D | H |
| 654c | H | H | H | D |
| 656c | D | D | H | H |
| 657c | D | H | D | H |
| 658c | D | H | H | D |
| 660c | H | D | D | H |
| 661c | H | D | H | D |
| 663c | H | H | D | D |
| 666c | D | D | D | H |
| 667c | D | D | H | D |
| 669c | D | H | D | D |
| 672c | H | D | D | D |
| 676c | D | D | D | D |

or a pharmaceutically acceptable salt thereof.

In some embodiments, the compound is a compound of Formula 5, wherein X^{2a} and X^{2b} are the same; Y^{6a} and Y^{6b} are each H; Y^{7a} and Y^{7b} are the same; Y⁸ is D; and the compound is selected from any one of the compounds set forth in Table 5a (below):

TABLE 5
Exemplary Embodiments of Formula 5

| | | Each X^{2a}/ | Each Y^{7a}/ | |
|---|---|---|---|---|
| Compound # | Each X¹ | X^{2b} | Y^{7b} | R⁴ |
| 700 | H | H | H | CH₃ |
| 701 | D | H | H | CH₃ |
| 702 | H | D | H | CH₃ |
| 703 | H | H | D | CH₃ |
| 704 | H | H | H | CD₃ |
| 705 | D | D | H | CH₃ |
| 706 | D | H | D | CH₃ |
| 707 | D | H | H | CD₃ |
| 708 | H | D | D | CH₃ |
| 709 | H | D | H | CD₃ |
| 710 | H | H | D | CD₃ |
| 711 | D | D | D | CH₃ |
| 712 | D | D | H | CD₃ |
| 713 | D | H | D | CD₃ |
| 714 | H | D | D | CD₃ |
| 715 | D | D | D | CD₃ |

or a pharmaceutically acceptable salt thereof.

In some embodiments, the compound is a compound of Formula 6, wherein X^{2a} and X^{2b} are the same; Y^{6a} and Y^{6b} are each H; Y^{7a} and Y^{7b} are the same; Y⁸ is D; Y^{9a} and Y^{9b} are the same; Y¹⁰ is D; R⁴ and R⁵ are the same; and the compound is selected from any one of the compounds set forth in Table 6 (below):

TABLE 6
Exemplary Embodiments of Formula 6

| | | Each X^{2a}/ | Each Y^{7a}/ | Each Y^{9a}/ | |
|---|---|---|---|---|---|
| Compound # | Each X¹ | X^{2b} | Y^{7b} | Y^{9b} | R⁴/R⁵ |
| 720 | H | H | H | H | CH₃ |
| 721 | D | H | H | H | CH₃ |
| 722 | H | D | H | H | CH₃ |
| 723 | H | H | D | H | CH₃ |
| 724 | H | H | H | D | CH₃ |
| 725 | H | H | H | H | CD₃ |
| 726 | D | D | H | H | CH₃ |
| 727 | D | H | D | H | CH₃ |
| 728 | D | H | H | D | CH₃ |
| 729 | D | H | H | H | CD₃ |
| 730 | H | D | D | H | CH₃ |
| 731 | H | D | H | D | CH₃ |
| 732 | H | D | H | H | CD₃ |
| 733 | H | H | D | D | CH₃ |
| 734 | H | H | D | H | CD₃ |
| 735 | H | H | H | D | CD₃ |
| 736 | D | D | D | H | CH₃ |
| 737 | D | D | H | D | CH₃ |
| 738 | D | D | H | H | CD₃ |
| 739 | D | H | D | D | CH₃ |
| 740 | D | H | D | H | CD₃ |
| 741 | D | H | H | D | CD₃ |
| 742 | H | D | D | D | CH₃ |
| 743 | H | D | D | H | CD₃ |
| 744 | H | D | H | D | CD₃ |
| 745 | H | H | D | D | CD₃ |
| 746 | D | D | D | D | CH₃ |
| 747 | D | D | D | H | CD₃ |
| 748 | D | D | H | D | CD₃ |
| 749 | D | H | D | D | CD₃ |
| 750 | H | D | D | D | CD₃ |
| 751 | D | D | D | D | CD₃ |

or a pharmaceutically acceptable salt thereof.

In some embodiments, the compound is a compound of Formula 7, wherein X^{2a} and X^{2b} are the same; Y^{6a} and Y^{6b} are each H; Y^{7a} and Y^{7b} are the same; Y⁸ is D; Y^{11a} and Y^{11b} are the same; R⁴ and R⁶ are the same; and the compound is selected from any one of the compounds set forth in Table 7 (below):

TABLE 7
Exemplary Embodiments of Formula 7

| | | Each X^{2a}/ | Each Y^{7a}/ | Each Y^{11a}/ | |
|---|---|---|---|---|---|
| Compound # | Each X¹ | X^{2b} | Y^{7b} | Y^{11b} | R⁴/R⁶ |
| 760 | H | H | H | H | CH₃ |
| 761 | D | H | H | H | CH₃ |
| 762 | H | D | H | H | CH₃ |
| 763 | H | H | D | H | CH₃ |
| 764 | H | H | H | D | CH₃ |
| 765 | H | H | H | H | CD₃ |
| 766 | D | D | H | H | CH₃ |
| 767 | D | H | D | H | CH₃ |
| 768 | D | H | H | D | CH₃ |
| 769 | D | H | H | H | CD₃ |
| 770 | H | D | D | H | CH₃ |
| 771 | H | D | H | D | CH₃ |
| 772 | H | D | H | H | CD₃ |
| 773 | H | H | D | D | CH₃ |
| 774 | H | H | D | H | CD₃ |
| 775 | H | H | H | D | CD₃ |
| 776 | D | D | D | H | CH₃ |
| 777 | D | D | H | D | CH₃ |
| 778 | D | D | H | H | CD₃ |
| 779 | D | H | D | D | CH₃ |
| 780 | D | H | D | H | CD₃ |
| 781 | D | H | H | D | CD₃ |
| 782 | H | D | D | D | CH₃ |
| 783 | H | D | D | H | CD₃ |
| 784 | H | D | H | D | CD₃ |
| 785 | H | H | D | D | CD₃ |
| 786 | D | D | D | D | CH₃ |
| 787 | D | D | D | H | CD₃ |
| 788 | D | D | H | D | CD₃ |
| 789 | D | H | D | D | CD₃ |
| 790 | H | D | D | D | CD₃ |
| 791 | D | D | D | D | CD₃ |

or a pharmaceutically acceptable salt thereof.

In some embodiments, the compound is a compound of Formula III, wherein R^{a} is H and R^{b} is H; and the compound is selected from any one of the compounds set forth in Table 8 (below):

TABLE 8
Exemplary Embodiments of Formula III

| Compound # | Each Z¹ | Z² | Each Z³ | Each Z⁴ |
|---|---|---|---|---|
| 800 | D | H | H | H |
| 801 | H | D | H | H |
| 802 | H | H | D | H |
| 803 | H | H | H | D |
| 804 | D | D | H | H |
| 805 | D | H | D | H |
| 806 | D | H | H | D |
| 807 | H | D | D | H |
| 808 | H | D | H | D |
| 809 | H | H | D | D |
| 810 | D | D | D | H |
| 811 | D | D | H | D |
| 812 | D | H | D | D |
| 813 | H | D | D | D |
| 814 | D | D | D | D |

or a pharmaceutically acceptable salt thereof.

In some embodiments, the compound is a compound of Formula III, wherein R^{a} is H and R^{b} is H; and the compound is selected from any one of the compounds set forth in Table 8a (below):

TABLE 8a
Exemplary Embodiments of Formula III

| Compound # | Each Z¹ | Z² | Each Z³ | Each Z⁴ |
|---|---|---|---|---|
| 804 | D | D | H | H |
| 805 | D | H | D | H |
| 806 | D | H | H | D |
| 807 | H | D | D | H |
| 809 | H | H | D | D |
| 810 | D | D | D | H |
| 811 | D | D | H | D |
| 812 | D | H | D | D |
| 813 | H | D | D | D |
| 814 | D | D | D | D |

or a pharmaceutically acceptable salt thereof.

In some embodiments, the compound is a compound of Formula IIIa, wherein R^{a} is H and R^{b} is H; and the compound is selected from any one of the compounds set forth in Table 9 (below):

TABLE 9
Exemplary Embodiments of Formula IIIa

| Compound # | Each Z¹ | Z² | Each Z³ | Each Z⁴ |
|---|---|---|---|---|
| 800a | D | H | H | H |
| 801a | H | D | H | H |
| 802a | H | H | D | H |
| 803a | H | H | H | D |
| 804a | D | D | H | H |
| 805a | D | H | D | H |
| 806a | D | H | H | D |
| 807a | H | D | D | H |
| 808a | H | D | H | D |
| 809a | H | H | D | D |
| 810a | D | D | D | H |
| 811a | D | D | H | D |
| 812a | D | H | D | D |
| 813a | H | D | D | D |
| 814a | D | D | D | D |

or a pharmaceutically acceptable salt thereof.

In some embodiments, the compound is a compound of Formula IIIa, wherein R^{a} is H and R^{b} is H; and the compound is selected from any one of the compounds set forth in Table 9a (below):

TABLE 9a
Exemplary Embodiments of Formula IIIa

| Compound # | Each Z¹ | Z² | Each Z³ | Each Z⁴ |
|---|---|---|---|---|
| 804a | D | D | H | H |
| 805a | D | H | D | H |
| 806a | D | H | H | D |
| 807a | H | D | D | H |
| 809a | H | H | D | D |
| 810a | D | D | D | H |
| 811a | D | D | H | D |
| 812a | D | H | D | D |
| 813a | H | D | D | D |
| 814a | D | D | D | D |

or a pharmaceutically acceptable salt thereof.

In some embodiments, the compound is selected from any one of the Compounds set forth in Table 1a, Table 1aa, Table 1b, Table 1bb, Table 2a, Table 2b, Table 3a, Table 3b, Table 4a, Table 4b, Table 4c, Table 5, Table 6, or Table 7 (above), or a pharmaceutically acceptable salt thereof; wherein any atom not designated as deuterium is present at its natural isotopic abundance.

In some embodiments, the compound is selected from any one of the Compounds set forth in Table 8, Table 8a, Table 9, or Table 9a (above), or a pharmaceutically acceptable salt thereof; wherein any atom not designated as deuterium is present at its natural isotopic abundance.

The synthesis of compounds of Formula I, I′, Ia, Ib, Ic, and II and Formula 2-7 may be readily achieved by synthetic chemists of ordinary skill by reference to the Exemplary Synthesis and Examples disclosed herein, using appropriate starting materials and reagents. Relevant procedures analogous to those of use for the preparation of compounds of Formula I, I′, Ia, Ib, Ic, II, 2, 3, 4, 5, 6, 7 and intermediates thereof are disclosed, for instance in Chinese Patent Application No. CN107162893, PCT publication WO2014/140308, and Seebach, D. et al., Helvitica Chimica Acta (1988), 71(1): 155-167.

The synthesis of compounds of Formula III and IIIa may be readily achieved by synthetic chemists of ordinary skill by reference to the Exemplary Synthesis and Examples disclosed herein, using appropriate starting materials and reagents. For example, intermediates 21, 23, 24, 25, 28, 29, 31, 32, 35, 36, 42 and 44 may be converted to compounds of Formula III or IIIa through known synthetic methods. Relevant procedures analogous to those of use for the preparation of compounds of Formula III, IIIa and intermediates thereof are disclosed, for instance in Kuriyama, W. et al., Adv Synth & Catalysis (2010), 352(1), 92-96; Larcheveque, M., Synth Comm (1991), 21(22), 2295-300; and PCT publication WO2019/147503.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure.

### Exemplary Synthesis

A convenient method for synthesizing compounds of Formula Ib and Ic is depicted in Scheme 1.

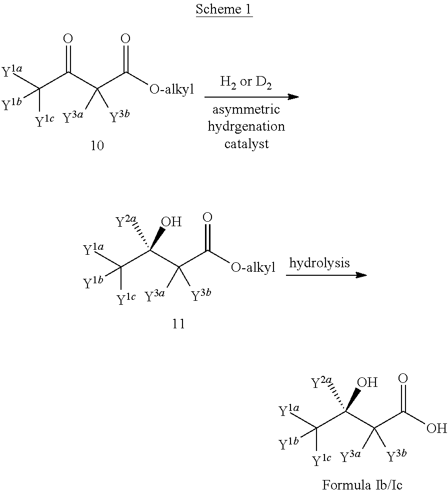

As depicted in Scheme 1, and in a manner analogous to that described in CN107162893, appropriately deuterated compounds of Formula Ib and Ic may be prepared from appropriately deuterated acetoacetate esters (10) through asymmetric hydrogenation using D₂ or H₂.

Compounds of Formula Ia, wherein R₂ is

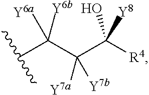

may be prepared in a manner analogous to that described in WO2014140308 from compounds of Formula Ic.

Compounds of Formula Ia, wherein R₁ is

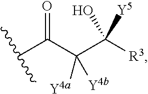

may be prepared from compounds of Formula Ic in a manner analogous to that described in Seebach, D. et al., Helvitica Chimica Acta (1988), 71(1): 155-167, 1988.

Compounds of Formula II may be prepared from appropriately deuterated starting materials such as acetoacetate esters (10) in a manner analogous to that described in U.S. Pat. No. 5,126,373, US 2012006411 or US 20160108442. Additionally, compounds of Formula II, 5, 6 and 7 may be readily prepared from compounds of Formula Ib/Ic by oxidation with Collins reagent, pyridinium chlorochromate (PCC), pyridininium dichromacte (PDC) as described by Luzzio, F. A., Org. React., 53, 1998. Alternatively, they may conveniently be prepared from compounds of Formula Ib/Ic by oxidation with Dess-Martin periodinane as described in Dess, D. B.; Martin, J. C. J. Am. Chem. Soc., 1991, 113 (19) 7277-7287, or by Swern oxidation as described in Omura, K.; Swern, D. Tetrahedron. (1978), 34 (11), 1651-1660.

Compounds of Formula III and IIIa may be prepared from appropriately deuterated starting materials such as β-hydroxy esters (11) by reduction with appropriately deuterated reagents analogously to as described in Kuriyama, W. et al., Adv Synth & Catalysis (2010), 352(1), 92-96; Larcheveque, M., Synth Comm (1991), 21(22), 2295-300; and PCT publication WO 2019147503.

Certain compounds of Formulae I, I′, Ia, Ib and Ic are known and in some cases are commercially available; otherwise compounds of Formula I, I′, Ia, Ib and Ic may be prepared according to methods known in the art.

Certain appropriately deuterated acetoacetate esters (10) are known and may be prepared according to methods known in the art.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I, I′, Ia, Ib and Ic and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene, T W et al., Protective Groups in Organic Synthesis, 3^{rd} Ed., John Wiley and Sons (1999); Fieser, L et al., Fieser and Fieser's Reagents for Organic Synthesis, John Wiley and Sons (1994); and Paquette, L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pharmaceutical compositions comprising an effective amount of a compound of Formula I, I′, Ia, Ib, Ic, or II (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

The invention also provides pharmaceutical compositions comprising an effective amount of a compound of Formula III, or IIIa (e.g., including any of the formulae herein), or a pharmaceutically acceptable salt of said compound; and a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium tri silicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States Patent Publication Nos. 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In another embodiment, a composition of this invention further comprises one or more additional therapeutic agents. The additional therapeutic agent may be selected from any compound or therapeutic agent known to have or that demonstrates advantageous properties when administered with a compound having the same mechanism of action as DBHB.

In certain embodiments, the additional therapeutic agent is an agent useful in the treatment of a disease or condition selected from a neurological or neuropsychiatric condition including, but not limited to, Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, eating disorders including anorexia nervosa and bulimia nervosa, type 2 diabetes, insulin resistance, and coronary artery disease.

In certain embodiments, a pharmaceutical composition containing a deuterated analog of DBHB (or other compound described herein) can be administered to a patient suffering from schizophrenia along with, or in sequence with, an art-known additional therapeutic agent for treating schizophrenia (e.g., olanzapine, clozapine, haloperidol, and the like). Such pharmaceutical compositions are included within the invention. In general, the antipsychotic therapeutic typically is administered at a dosage of 0.25-5000 mg/day (e.g., 5-1000 mg/day)). “Typical” antipsychotics are conventional antipsychotics such as phenothiazine, butryophenones, thioxantheses, dibenzoxazepines, dihydroindolones, and diphenylbutylpiperidines. “Atypical” antipsychotics are a newer generation of antipsychotics which generally act on the dopamine D₂ and 5HT₂ serotonin receptor and have high levels of efficacy and a benign extrapyramidal symptom side effect profile. Examples of typical antipsychotics include chlorpromazine, thioridazine, mesoridazine, fluphenazine, perphenazine, trifluoperazine, thiothixene, haloperidol, loxapine, molindone, acetophenazine, chlorprothixene, droperidol, and pimozide. Examples of atypical antipsychotics include bolanserin, clozapine, risperidone, olanzapine, cariprazine, asenapine, lurasidone, brexpiprazole, lumateperone, aripiprazole, aripiprazole lauroxil, iloperidone, paliperidone, ziprasidone, and quetiapine. Depot antipsychotics also can be used, e.g., haloperidol decanoate, fluphenazine decanoate, and fluphenazine enanthate. Additional antipsychotics include butaperazine, carphenazine, remoxipride, piperacetazine, and sulpiride.

In another embodiment, the invention provides separate dosage forms of a compound of this invention and one or more of any of the above-described additional therapeutic agents, wherein the compound and additional therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat the target disorder. As described above, the dosing regimen can include one or more additional therapeutic agents (e.g., where the compound or composition of the invention is used in a combination (e.g., when a compound or composition of the invention is used as an adjunctive therapy).

The term “subject in need thereof,” refers to a subject having or being diagnosed with a disease or condition selected from Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, eating disorders including anorexia nervosa and bulimia nervosa, type 2 diabetes, insulin resistance, and coronary artery disease.

The interrelationship of dosages for animals and humans (based on milligrams per meter squared of body surface) is described in Freireich et al., Cancer Chemother. Rep., 1966, 50: 219. Body surface area may be approximately determined from height and weight of the subject. See, e.g., Scientific Tables, Geigy Pharmaceuticals, Ardsley, N.Y., 1970, 537.

In certain embodiments, an effective amount of a compound of Formula I, Formula I′ or Formula II can range from 1 to 60 g/day, or from 5 to 30 g/day, or from 10 to 20 g/day. In certain embodiments, an effective amount of a compound of Formula I, I′, Ia, Ib, Ic, or II can range from 100 mg to 1 g/day. In certain embodiments, an effective amount of a compound of Formula I, I′, Ia, Ib, Ic, or II can range from 1 to 10 g/day. In certain embodiments, an effective amount of a compound of Formula I, I′, Ia, Ib, Ic, or II can range from 10 to 25 g/day.

In certain embodiments, an effective amount of a compound of Formula III, or Formula IIIa can range from 1 to 60 g/day, or from 5 to 30 g/day, or from 10 to 20 g/day. In certain embodiments, an effective amount of a compound of Formula III, or IIIa can range from 100 mg to 1 g/day. In certain embodiments, an effective amount of a compound of Formula III, or IIIa can range from 1 to 10 g/day. In certain embodiments, an effective amount of a compound of Formula III, or IIIa can range from 10 to 25 g/day.

In certain embodiments, an effective amount of a compound of Formula I, I′, Ia, Ib, Ic, or II can range from 30 milligrams per kilogram body weight per day (mg/kg/day) to 900 mg/kg/day, or from 140 mg/kg/day to 710 mg/kg/day, or from 420 mg/kg/day to 900 mg/kg/day, or from 80 mg/kg/day to 420 mg/kg/day, or from 240 mg/kg/day to 710 mg/kg/day, or from 60 mg/kg/day to 300 mg/kg/day, or from 150 mg/kg/day to 300 mg/kg/day. In certain embodiments, an effective amount of a compound of Formula I, I′, Ia, Ib, Ic, or II can range from 10 mg/kg/day to 150 mg/kg/day, or 10 mg/kg/day to 120 mg/kg/day, or 10 mg/kg/day to 90 mg/kg/day. The compound of the invention can be administered once daily, twice daily or three times daily.

In certain embodiments, an effective amount of a compound of Formula III, or IIIa can range from 30 milligrams per kilogram body weight per day (mg/kg/day) to 900 mg/kg/day, or from 140 mg/kg/day to 710 mg/kg/day, or from 420 mg/kg/day to 900 mg/kg/day, or from 80 mg/kg/day to 420 mg/kg/day, or from 240 mg/kg/day to 710 mg/kg/day, or from 60 mg/kg/day to 300 mg/kg/day, or from 150 mg/kg/day to 300 mg/kg/day. In certain embodiments, an effective amount of a compound of Formula III, or Ma can range from 10 mg/kg/day to 150 mg/kg/day, or 10 mg/kg/day to 120 mg/kg/day, or 10 mg/kg/day to 90 mg/kg/day. The compound of the invention can be administered once daily, twice daily or three times daily.

In certain embodiments, an effective amount of a compound of Formula I, I′, Ia, Ib, Ic, or II can range from 1 to 60 g/day, or from 5 to 30 g/day, or from 10 to 20 g/day.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for a compound of Formula I, I′, Ia, Ib, Ic, or II.

For pharmaceutical compositions that comprise one or more additional therapeutic agents, an effective amount of the additional therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

Some of the additional therapeutic agents referenced above may act synergistically with the compounds of this invention. When this occurs, it will allow the effective dosage of the additional therapeutic agent and/or the compound of this invention to be reduced from that required in a monotherapy. This has the advantage of minimizing toxic side effects of either the additional therapeutic agent of a compound of this invention, synergistically improving efficacy, improving ease of administration or use and/or reduced overall expense of compound preparation or formulation.

### Methods of Treatment

In another aspect, the invention provides therapeutic methods.

In one embodiment, the invention provides a method of treating disorders responsive to increases levels of BDNF, the method comprising administering to a subject in need thereof an effective amount of a pharmaceutical composition of this invention.

In another embodiment, the invention provides a method of treating a neurological or neuropsychiatric condition including, but not limited to, Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, and eating disorders including anorexia nervosa and bulimia nervosa. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula 2, 3, or 4. In one embodiment, the compound of Formula II is a compound of Formula 5, 6 or 7.

In another embodiment, the invention provides a method of treating type 2 diabetes, insulin resistance, coronary artery disease, or of regulating or enhancing pools of adult stem cells. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6 or 7.

In certain more particular embodiments, the invention provides a method of treating major depressive disorder, major depressive disorder with mixed features, bipolar disorder, bipolar mania, bipolar depression or treatment-refractory depression. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6 or 7.

In certain more particular embodiments, the invention provides a method of treating mild cognitive impairment, cognitive deficits in Parkinson's disease, cognitive deficits in depression, cognition deficits associated with Huntington's disease, subjective cognitive decline or age-related memory loss. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6 or 7.

In certain more particular embodiments, the invention provides a method of treating schizophrenia. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6 or 7.

In certain more particular embodiments, the invention provides a method of treating epilepsy or controlling epileptic seizures. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6, or 7.

In another aspect, the invention provides a method of increasing BDNF, the method comprising contacting a cell with a compound of this invention, such that BDNF in the cell is increased. In one embodiment, the compound of Formula Ia is a compound of Formula Ib, 2, 3, or 4. In one embodiment, the compound of Formula II is a compound of Formula 5, 6, or 7. In one embodiment, the compound is a compound of Formula III or IIIa.

In another aspect, the invention provides a method of antagonizing histone deacetylases HDAC2 and HDAC3 in a cell, comprising contacting a cell with one or more compounds of Formula I, I′, Ia, Ib, Ic, or II herein, or a pharmaceutically acceptable salt thereof. In some embodiments, the cell is contacted in vitro. In some embodiments, the cell is contacted in vivo. In some embodiments, the cell is contacted ex vivo. In one embodiment, the compound of Formula Ia is a compound of Formula Ib, 2, 3, or 4. In one embodiment, the compound of Formula II is a compound of Formula 5, 6, or 7.

In another aspect, the invention provides a method of upregulating the transcription factor forkhead box O-3 (FoxO3).

In another aspect, the invention provides a method of upregulating FoxO3 in a cell, comprising contacting a cell with one or more compounds of Formula I, I′, Ia, Ib, Ic, or II herein, or a pharmaceutically acceptable salt thereof. In some embodiments, the cell is contacted in vitro. In some embodiments, the cell is contacted in vivo. In some embodiments, the cell is contacted ex vivo. In one embodiment, the compound of Formula Ia is a compound of Formula Ib, 2, 3, or 4. In one embodiment, the compound of Formula II is a compound of Formula 5, 6, or 7.

In another aspect, the invention provides a method of treating disorders responsive to increases levels of BDNF, the method comprising the step of administering to a subject in need thereof an effective amount of a compound of Formula I, I′, Ia, Ib, Ic, or II of a pharmaceutical composition of this invention, such that the disease or condition is treated. In one embodiment, the compound of Formula Ia is a compound of Formula 2, 3, or 4. In one embodiment, the compound of Formula II is a compound of Formula 5, 6, or 7.

In some embodiments, the invention provides a method for treating neurological or neuropsychiatric conditions including, but not limited to, Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, and eating disorders including anorexia nervosa and bulimia nervosa, and the like. In certain embodiments, the method of this invention is used to treat a disease or condition selected from mild cognitive impairment, cognitive deficits in Parkinson's disease, cognitive deficits in depression, cognition deficits associated with Huntington's disease, subjective cognitive decline and age-related memory loss in a subject in need thereof. In certain embodiments, the method of this invention is used to treat a disease or condition selected from major depressive disorder, major depressive disorder with mixed features, bipolar disorder, bipolar mania, bipolar depression and treatment-refractory depression in a subject in need thereof. In certain embodiments, the method of this invention is used to treat epilepsy in a subject in need thereof. The method comprises administering to the subject in need thereof an effective amount of a compound of Formula I, I′, Ia, Ib, Ic, or II or a pharmaceutical composition comprising a compound of Formula I, I′, Ia, Ib, Ic, or II, such that the disease or condition is treated. In one embodiment, the compound of Formula Ia is a compound of Formula Ib, 2, 3, or 4. In one embodiment, the compound of Formula II is a compound of Formula 5, 6, or 7.

In another embodiment, the invention provides a method of treating all of the above-disclosed diseases or conditions, the method comprising administering to the subject in need thereof an effective amount of a compound of Formula III, or IIIa or a pharmaceutical composition comprising a compound of Formula III, or IIIa, such that the disease or condition is treated.

In certain embodiments, the method of treatment comprises administering to a subject in need thereof a pharmaceutical composition comprising a compound of Formula I, I′, Ia, Ib, Ic, or II or a pharmaceutically acceptable salt thereof, and a pharmaceutically acceptable carrier, wherein the amount of the compound of Formula I, I′, Ia, Ib, Ic, or II administered per day is in the range of 1 mg/kg to 200 mg/kg (i.e., 1 mg per kilogram of body weight of the subject to 200 per kilogram of body weight of the subject). In one embodiment, the compound of Formula Ia is a compound of Formula Ib, 2, 3, or 4. In one embodiment, the compound of Formula II is a compound of Formula 5, 6, or 7.

In certain embodiments, the compound of Formula I, I′, Ia, Ib, Ic, or II or a pharmaceutical composition comprising a compound of Formula I, I′, Ia, Ib, Ic, or II is administered once per day. In other embodiments, the compound of Formula I, I′, Ia, Ib, Ic, or II or a pharmaceutical composition comprising a compound of Formula I, I′, Ia, Ib, Ic, or II is administered twice per day. In yet other embodiments, the compound of Formula I, I′, Ia, Ib, Ic, or II or a pharmaceutical composition comprising a compound of Formula I, I′, Ia, Ib, Ic, or II is administered three times per day. In yet other embodiments, the compound of Formula I, I′, Ia, Ib, Ic, or II or a pharmaceutical composition comprising a compound of Formula I, I′, Ia, Ib or Ic II is administered four times per day. In one embodiment, the compound of Formula Ia is a compound of Formula Ib, 2, 3, or 4. In one embodiment, the compound of Formula II is a compound of Formula 5, 6, or 7.

In certain embodiments, an effective amount of a compound of Formula I, I′, Ia, Ib, Ic, or II can range from 1 to 60 g/day, or from 5 to 30 g/day, or from 10 to 20 g/day. In certain embodiments, an effective amount of a compound of Formula I, I′, Ia, Ib, Ic, or II can range from 100 mg to 1 g/day. In certain embodiments, an effective amount of a compound of Formula I, I′, Ia, Ib, Ic, or II can range from 1 to 10 g/day. In one embodiment, the compound of Formula Ia is a compound of Formula Ib, 2, 3, or 4. In one embodiment, the compound of Formula II is a compound of Formula 5, 6, or 7.

In certain embodiments, an effective amount of a compound of Formula I, I′, Ia, Ib, Ic, or II can range from 30 milligrams per kilogram body weight per day (mg/kg/day) to 900 mg/kg/day, or from 140 mg/kg/day to 710 mg/kg/day, or from 420 mg/kg/day to 900 mg/kg/day, or from 80 mg/kg/day to 420 mg/kg/day, or from 240 mg/kg/day to 710 mg/kg/day, or from 60 mg/kg/day to 300 mg/kg/day, or from 150 mg/kg/day to 300 mg/kg/day. In certain embodiments, an effective amount of a compound of Formula I, I′, Ia, Ib, Ic, or II can range from 10 mg/kg/day to 150 mg/kg/day, or 10 mg/kg/day to 120 mg/kg/day, or 10 mg/kg/day to 90 mg/kg/day. In one embodiment, the compound of Formula Ia is a compound of Formula Ib, 2, 3, or 4. In one embodiment, the compound of Formula II is a compound of Formula 5, 6, or 7.

Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject in need thereof one or more additional therapeutic agents. The choice of additional therapeutic agent may be made from any additional therapeutic agent known to be useful for co-administration with a compound of Formula I, I′, Ia, Ib, Ic, or II. The choice of additional therapeutic agent is also dependent upon the particular disease or condition to be treated. Examples of additional therapeutic agents that may be employed in the methods of this invention are those set forth above for use in combination compositions comprising a compound of this invention and an additional therapeutic agent.

The term “co-administered” as used herein means that the additional therapeutic agent may be administered together with a compound of this invention as part of a single dosage form (such as a composition of this invention comprising a compound of the invention and an additional therapeutic agent as described above) or as separate, multiple dosage forms. Alternatively, the additional agent may be administered prior to, consecutively with, or following the administration of a compound of this invention. In such combination therapy treatment, both the compounds of this invention and the additional therapeutic agent(s) are administered by conventional methods. The administration of a composition of this invention, comprising both a compound of the invention and an additional therapeutic agent, to a subject does not preclude the separate administration of that same therapeutic agent, any other additional therapeutic agent or any compound of this invention to said subject at another time during a course of treatment.

Effective amounts of these additional therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the additional therapeutic agent's optimal effective-amount range.

In one embodiment of the invention, where an additional therapeutic agent is administered to a subject, the effective amount of the compound of this invention is less than its effective amount would be where the additional therapeutic agent is not administered. In another embodiment, the effective amount of the additional therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I, I′, Ia, Ib, Ic, or II alone or together with one or more of the above-described additional therapeutic agents in the manufacture of a medicament, either as a single composition or as separate dosage forms, for treatment in a subject of a disease, disorder or symptom set forth above. In one embodiment of this aspect, the compound of Formula Ia is a compound of Formula Ib, 2, 3, or 4. In one embodiment, the compound of Formula II is a compound of Formula 5, 6, or 7. Another aspect of the invention is a compound of Formula I, I′, Ia, Ib, Ic, or II for use in the treatment in a subject of a disease, disorder or symptom thereof delineated herein. In one embodiment of this aspect, the compound of Formula Ia is a compound of Formula Ib, 2, 3, or 4. In one embodiment, the compound of Formula II is a compound of Formula 5, 6, or 7.

In yet another aspect, the invention provides the use of a compound of Formula III, or Ma alone or together with one or more of the above-described additional therapeutic agents in the manufacture of a medicament, either as a single composition or as separate dosage forms, for treatment in a subject of a disease, disorder or symptom set forth above.

### EXAMPLES

### Example 1. (R)-3-Hydroxybutanoic-3-d₁ Acid (Compound 100)

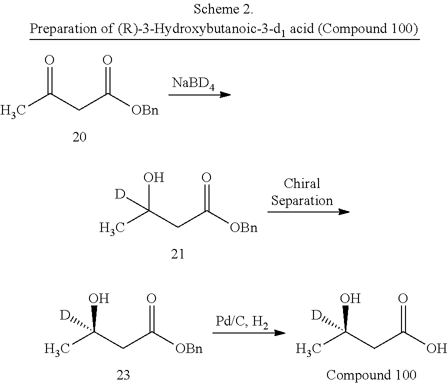

Step 1. Benzyl 3-hydroxybutanoate-3-d₁ (21). Sodium borodeuteride (Aldrich, 98 atom % D) (0.33 g, 7.8 mmol) was added to a solution of benzyl acetoacetate (5.0 g, 26.0 mmol) in 5:1 mixture of tetrahydrofuran and water (312 mL) at 0° C. The reaction mixture was stirred at the same temp for 3 h, then diluted with water (250 mL) and extracted with MTBE (3×200 mL). The combined organic layers were washed with saturated brine (200 mL) and concentrated under reduced pressure. The crude product was purified by flash chromatography (Interchim system, SorbTech 80 g silica gel column, gradient of 0-40% ethyl acetate-hexanes) to afford 21 (4.7 g, 99% yield) as a light yellow oil.

Chiral separation of 21, isolation of benzyl (R)-3-hydroxybutanoate-3-d₁ (23): The racemic 21 (4.7 g) was separated by chiral SFC (method: AD-H column, 3×25 cm, eluting with 11% methanol/CO₂ at 100 bar as the mobile phase, 70 mL/min). The chiral SFC elution times were (R) isomer 23: 4.07 min, (S) isomer: 3.79 min. The (R) isomer 23 was obtained as a colorless oil (2 g).

Chiral HPLC analytical method: Chiralpak, OD-H 250×4.6 mm, 10 μm; 90:10 hexane: i-propanol; flow 1.0 mL/min; Wavelength: 254 nm; (R) isomer 23: 6.88 min, (S) isomer: 7.70 min. The desired enantiomer 23 was obtained in 99% ee.

Step 2. (R)-3-Hydroxybutanoic-3-d acid (Compound 100): A solution of 23 (0.5 g, 2.6 mmol) in ethyl acetate (20 mL) was subjected to hydrogenation at a pressure of 30 psi H₂ in the presence of 10% palladium on carbon (0.10 g, 50% wet) for 2 h. The reaction mixture was filtered through a syringe filter, concentrated under reduced pressure and lyophilized from water to give Compound 100 as a white solid (120 mg, 44% yield).

¹H NMR (CDCl₃, 400 MHz): δ 1.26 (s, 3H), 2.45-2.57 (m, 2H), 6.30 (bs, 2H). GC (method: Phenomenex ZB-1MS column, 30 m×0.25 mm, 0.25 um; start temp 50° C., ramp 20° C./min to 300° C., hold for 5 min): retention time: 3.7 min; purity 99.9%. LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (EI): m/z=104.1 ([M−H]⁺).

### Example 2. (R)-3-hydroxybutanoic-3,4,4,4-d₄ Acid (Compound 102)

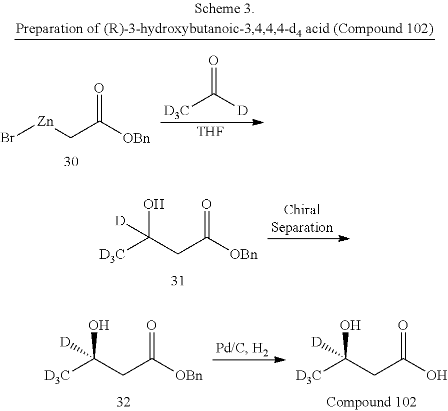

Step 1. Benzyl 3-hydroxybutanoate-3,4,4,4-d₄ (31): A solution of acetaldehyde-d₄ (CDN, 99.5 atom % D, 1.0 g, 20.8 mmol) in anhydrous THF (20 mL) was added dropwise to commercially available 2-benzyloxy-2-oxoethylzinc bromide (0.5 M in ether, 58.2 mL, 29.2 mmol) at 0° C. The reaction mixture was warmed to rt and stirred for 16 h, then concentrated under reduced pressure. The residue was adsorbed onto silica gel and purified by flash chromatography (Interchim system, SorbTech 40 g silica gel column, gradient of 0-30% ethyl acetate-hexanes) to afford 31 (3.3 g, 81% yield) as a clear oil.

¹H NMR (CDCl₃, 500 MHz): δ 2.45-2.57 (m, 2H), 2.90 (s, 1H), 5.16 (s, 2H), 7.32-7.40 (m, 5H). EI MS: m/z=199.1 ([M+H]⁺).

Chiral separation of 31, isolation of benzyl (R)-3-hydroxybutanoate-3,4,4,4-d₄ (32): The racemic 31 (3.3 g) was purified by chiral SFC (method: AD-H column, 3×25 cm, eluting with 11% methanol/CO₂ at 100 bar as the mobile phase, 70 mL/min). The chiral SFC elution times were: (R) isomer 32: 4.07 min, (S) isomer: 3.79 min. The (R) isomer 32 was further purified by flash chromatography (Interchim system, SorbTech 24 g silica gel column, gradient of 0-30% ethyl acetate-hexanes) to give 32 as a clear oil (0.87 g).

Chiral HPLC analytical method: Chiralpak, OD-H 250×4.6 mm, 10 μm; 90:10 hexane:i-propanol; flow 1.0 mL/min; Wavelength: 216 nm; (R) isomer 32: 7.14 min, (S) isomer: 8.15 min. The desired enantiomer 32 was obtained in >99% ee.

Step 2. (R)-3-hydroxybutanoic-3,4,4,4-d₄ acid (Compound 102): A solution of 32 (0.4 g, 2.0 mmol) in ethyl acetate (20 mL) was subjected to hydrogenation at a pressure of 30 psi H₂ in the presence of 10% palladium on carbon (0.08 g, 50% wet) for 2 h. The reaction mixture was filtered through a syringe filter, concentrated under reduced pressure, and lyophilized from acetonitrile/water to give Compound 102 (0.16 g, 74% yield) as a white solid.

¹H NMR (CDCl₃, 400 MHz): δ 2.45-2.57 (m, 2H), 5.50 (bs, 2H). LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (EI-MS): m/z=109.1 ([M−H]⁺).

### Example 3. (R)-3-hydroxybutanoic-2,2,3-d₃ Acid (Compound 104)

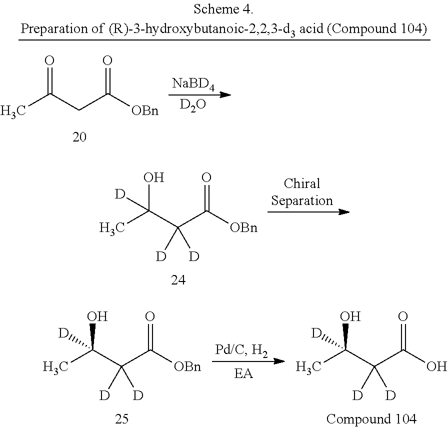

Step 1. Benzyl 3-hydroxybutanoate-2,2,3-d₃ (24): A mixture of ester 20 (3.0 g, 16 mmol) and D₂O (CIL, 99.9 atom % D, 6 mL) was stirred vigorously at 70° C. for 10 min. The mixture was cooled to rt and the D₂O decanted from the bi-phasic mixture. The deuterium exchange process was repeated three times. Additional D₂O (6 mL) was added to the oil, the reaction mixture was cooled to 0° C., and a solution of sodium borodeuteride (CIL, 99 atom % D, 0.21 g, 4.8 mmol, 0.3 equiv) in D₂O (3 mL) was added. The reaction mixture was warmed slowly to rt and stirred overnight. The D₂O was decanted to give crude 24 (3 g).

Chiral separation of 24, isolation of benzyl (R)-3-hydroxybutanoate-2,2,3-d₃ (25): The racemic 24 (3 g) was purified by chiral SFC (method: AD-H column, 3×25 cm, eluting with 9% methanol/CO₂ at 100 bar as the mobile phase, 70 mL/min). The chiral SFC elution times were: (R) isomer 25: 4.19 min, (S) isomer: 3.93 min. The (R) isomer 25 was obtained as a colorless oil, which was further purified by flash chromatography (Interchim system, SorbTech 80 g column, gradient of 0-30% ethyl acetate-hexanes) to afford 25 (0.6 g) as a clear oil.

Chiral HPLC analytical method: Chiralpak, OD-H 250×4.6 mm, 10 μm; 90:10 hexane: i-propanol; flow 1.0 mL/min; Wavelength: 254 nm; (R) isomer 25: 7.20 min, (S) isomer: 8.18 min. The desired enantiomer 25 was obtained in 99% ee.

Step 2. (R)-3-hydroxybutanoic-2,2,3-d₃ acid (Compound 104). A solution of ester 25 (0.6 g, 3.0 mmol) in ethyl acetate (20 mL) was subjected to hydrogenation at a pressure of 30 psi H₂ in the presence of 10% palladium on carbon (0.12 g, 50% wet) for 6 h. The reaction mixture was filtered via syringe filter, concentrated under reduced pressure, and lyophilized from water to give Compound 104 (106 mg, 32% yield).

¹H NMR (CDCl₃, 400 MHz): δ 1.25 (s, 3H), 6.25 (bs, 2H). 5%, 7% proton incorporation at each site alpha to the carboxylic acid moiety. LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (ES-API): m/z=108.1 ([M+H]⁺).

### Example 4. (R)-3-Hydroxybutanoic-2,2,3,4,4,4-d₆ Acid (Compound 105)

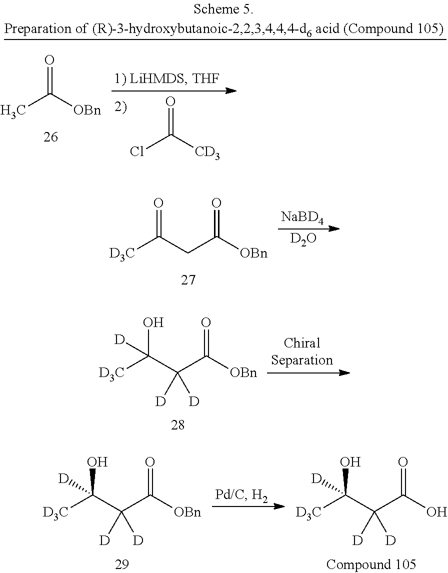

Step 1. Benzyl 3-oxobutanoate-4,4,4-d₃ (27): To a solution of benzyl acetate (3.0 g, 20 mmol) in anhydrous THF (10 mL) was added lithium bis(trimethyl-silyl)amide (1.0M in THF, 40 mL, 40 mmol). The resulting solution was cooled to −78° C. and stirred at the same temperature for 1 h. Acetyl chloride-d₃ (Aldrich, 99 atom % D, 1.63 g, 20 mmol) was added dropwise over 15 minutes and the solution was stirred for 2 h. The reaction mixture was warmed to rt, then quenched by slow addition of 10% aqueous HCl solution (7 mL) followed by water (25 mL). The layers were separated and the aqueous layer was extracted with diethyl ether (2×50 mL). The combined organic layers were washed with 10% aqueous HCl solution (50 mL), saturated sodium bicarbonate solution (50 mL) and saturated sodium chloride solution (50 mL). The organic layer was dried over sodium sulfate, filtered and concentrated under reduced pressure.

The crude product was purified by flash chromatography (Interchim system, SorbTech 40 g silica gel column, gradient of 5-30% ethyl acetate-hexanes) to afford 27 (3.1 g, 78% yield) as a pale yellow oil.

¹H NMR (CDCl₃, 500 Mhz): δ 3.50 (s, 2H), 5.18 (s, 2H), 7.32-7.40 (m, 5H). EI MS: m/z=195.1 ([M]⁺).

Step 2. Benzyl 3-hydroxybutanoate-2,2,3,4,4,4-d₆ (28): A bi-phasic mixture of ester 27 (3.1 g, 16 mmol) and D₂O (CIL, 99.9 atom % D, 15 mL) was stirred vigorously at 70° C. for 15 min, then the reaction mixture was concentrated under reduced pressure. This process was repeated three times. An additional portion of D₂O (5 mL) was added, and the reaction mixture was cooled to 0° C. A solution of sodium borodeuteride (Aldrich, 98 atom % D, 0.2 g, 4.8 mmol) in D₂O (5 mL) was added. The reaction mixture was warmed slowly to rt and stirred overnight. The aqueous layer was decanted to give crude 28 (3.1 g).

¹H NMR (CDCl₃, 500 Mhz): δ 5.16 (s, 2H), 7.32-7.40 (m, 5H). EI MS: m/z=200.1 ([M]⁺).

Chiral separation of 28, isolation of benzyl (R)-3-hydroxybutanoate-2,2,3,4,4,4-d₆ (29): The racemic 28 (3.1 g) was separated by chiral SFC (method: AD-H column, 3×25 cm, eluting with 8% methanol (0.1% diethylamine)/CO₂ at 100 bar as the mobile phase, 70 mL/min). The chiral SFC elution times were: (R) isomer 29: 3.83 min, (S) isomer: 3.65 min. The (R) isomer 29 was further purified by flash chromatography (Interchim system, SorbTech 40 g silica gel column, gradient of 0-30% ethyl acetate-hexanes) to give 29 as a clear oil (0.5 g).

Chiral HPLC analytical method: Chiralpak, AD-H 250×4.6 mm, 10 μm; 80:20 hexane:EtOH; flow 1.0 mL/min; Wavelength: 216 nm; (R) isomer 29: 9.41 min, (S) isomer: 11.23 min. The desired enantiomer 29 was obtained in >99% ee.

Step 3. (R)-3-Hydroxybutanoic-2,2,3,4,4,4-d₆ acid (Compound 105): A solution of 29 (0.4 g, 2.0 mmol) in ethyl acetate (40 mL) was subjected to hydrogenation at a pressure of 30 psi H₂ in the presence of 10% palladium on carbon (80 mg, 50% wet) for 6 h. The reaction mixture was filtered through a syringe filter, concentrated under reduced pressure and purified by flash chromatography (Interchim system, SorbTech 12 g silica gel column, gradient of 0-90% ethyl acetate-hexanes). Product fractions were concentrated under reduced pressure, then lyophilized from acetonitrile and water to afford Compound 105 (51 mg, 23% yield) as a white solid.

¹H NMR (CDCl₃, 400 MHz): δ 6.10 (bs, 2H). (3%, 4% residual H incorporation at each site alpha to the carboxylic acid moiety.) LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (ES-API): m/z=111.1 ([M+H]⁺).

### Example 5. (R)-3-hydroxybutanoic-4,4,4-d₃ Acid (Compound 202)

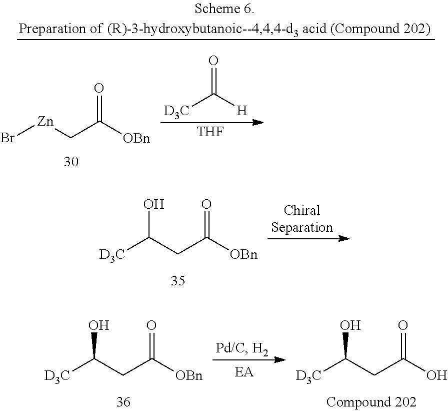

Step 1. Benzyl 3-hydroxybutanoate-4,4,4-d₃ (35): A solution of acetaldehyde-2,2,2-d₃ (98.2 atom % D, CDN) (1.0 g, 21.3 mmol) in anhydrous THF (20 mL) was added dropwise to commercially available 2-benzyloxy-2-oxoethylzinc bromide (0.5M in ether, 59.6 mL, 29.8 mmol) at 0° C. The reaction mixture was slowly warmed to rt, stirred for 16 h then concentrated under reduced pressure. The residue was adsorbed onto silica gel and purified by flash chromatography (Interchim system, SorbTech 40 g silica gel column, gradient of 0-30% ethyl acetate-hexanes) to afford 35 (4.1 g, 99% yield) as a light yellow oil.

¹H NMR (CDCl₃, 500 MHz): δ 2.45-2.57 (m, 2H), 2.90 (s, 1H), 4.21 (s, 1H), 5.16 (s, 2H), 7.32-7.40 (m, 5H). EI MS: m/z=198.1 ([M+H]⁺).

Chiral separation of 35, isolation of benzyl (R)-3-hydroxybutanoate-4,4,4-d₃ (36): The racemic 35 (4.1 g) was separated by chiral SFC (method: AD-H column, 3×25 cm, eluting with 11% methanol/CO₂ at 100 bar as the mobile phase, 70 mL/min). The chiral SFC elution times were: (R) isomer 36: 4.04 min, (S) isomer: 3.81 min. The (R) isomer 36 was obtained as a colorless oil (1.4 g).

Chiral HPLC analytical method: Chiralpak, OD-H 250×4.6 mm, 10 μm; 90:10 hexane: i-propanol with 0.1% TFA; flow 1.0 mL/min; Wavelength: 254 nm; (R) isomer 36: 6.84 min, (S) isomer: 7.67 min. The desired enantiomer 36 was obtained in 99.5% ee.

Step 2. (R)-3-Hydroxybutanoic-4,4,4-d₃ acid (Compound 202): A solution of 36 (0.4 g, 2 mmol) in ethyl acetate (20 mL) was subjected to hydrogenation at a pressure of 20 psi H₂ in the presence of 10% palladium on carbon (0.08 g, 50% wet) for 2 h. The mixture was filtered through a syringe filter and concentrated under reduced pressure. The residue was purified by flash chromatography (Interchim system, SorbTech 12 g silica gel column, gradient of 0-90% ethyl acetate-hexanes). Product fractions were concentrated under reduced pressure and the residue was lyophilized from acetonitrile and water to yield Compound 202 (77 mg, 30% yield) as a white solid.

¹H NMR (CDCl₃, 400 MHz): δ 2.45-2.57 (m, 2H), 4.25 (m, 1H), 5.50 (bs, 2H). LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (EI-MS): m/z=108.1 ([M+H]⁺).

### Example 6. (R)-3-Hydroxybutanoic-2,2-d₂ Acid (Compound 204)

Compound 204 was prepared according to Scheme 7 as described below.

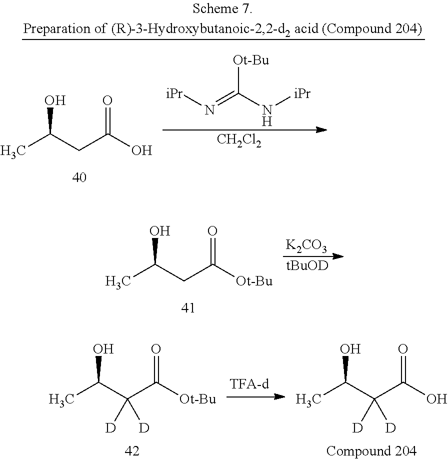

Step 1. tert-Butyl (R)-3-hydroxybutanoate (41): Commercially available tert-butyl (Z)—N,N′-diisopropyl-carbamimidate (28.9 g, 144 mmol) was added dropwise to a solution of 40 (5 g, 48.0 mmol) in dichloromethane (100 mL) at 5° C. The reaction mixture was stirred at rt for 65 h, with formation of a heavy white suspension. The suspension was filtered through a pad of Celite. The filtrate was concentrated under reduced pressure with additional precipitation of solid. The precipitate was filtered, and the filtrate was concentrated under reduced pressure. The crude residue was purified by flash chromatography (Interchim system, SorbTech 220 g silica gel column, gradient of 23-30% ethyl acetate-hexanes) to afford 41 (3.0 g, 39% yield) as a clear oil.

Step 2. tert-Butyl (R)-3-hydroxybutanoate-2,2-d₂ (42): Ester 41 (2 batches of 0.5 g each, 3.1 mmol) was dissolved in methanol-d (Aldrich, 99.3 atom % D, 2 mL) and then concentrated under reduced pressure. This process was repeated. Each residue was then treated with potassium carbonate (0.04 g, 0.31 mmol) in t-BuOD (CDN, 99.2 atom % D, 5 mL) at 100° C. overnight. ¹H NMR analysis of aliquots of the reaction mixtures indicated 32% (batch 1) and 20% (batch 2) proton signal remaining for the alpha-protons. The reaction mixtures were concentrated under reduced pressure. An additional portion of t-BuOD (5 mL) and potassium carbonate (0.1 equiv) were added to each reaction and the reaction mixtures were heated at 100° C. overnight, then concentrated under reduced pressure. This process was repeated for 5 cycles, at which point ¹H-NMR analysis indicated 2.5% and 2% proton remaining for the alpha-protons. Each reaction mixture was filtered and concentrated under reduced pressure to give 2 batches of crude 42 (0.17 g, 33% yield, 3% proton remaining; and 0.17 g, 33% yield, 4% proton remaining) as clear oils. This material was used without further purification.

¹H NMR (CDCl₃, 500 MHz): δ 1.20-1.21 (d, 3H), 1.47 (s, 9H), 2.30-2.38 (m, 3% to 4% proton incorporation), 4.11-4.15 (m, 1H).

Step 3. (R)-3-Hydroxybutanoic-2,2-d₂ acid (Compound 204): A mixture of 42 (0.16 g, 0.97 mmol) and trifluoroacetic acid-OD (Aldrich, 99.5 atom % D, 2.2 g, 19.3 mmol) was stirred at rt for 2 h. The reaction mixture was concentrated under reduced pressure at rt and then at 30° C. for 5 minutes. The crude material was purified by flash chromatography (Interchim system, SorbTech 12 g silica gel column, gradient of 0-90% ethyl acetate-hexanes) to afford Compound 204 (63 mg, 61% yield, 6.5% proton remaining) as a white solid.

¹H NMR (CDCl₃, 500 MHz): δ 1.25-1.27 (d, 3H), 4.21-4.25 (m, 1H), 5.95 (bs, 2H). (6.5% proton incorporation at each site alpha to the carboxylic acid moiety.) LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (EI-MS): m/z=107.1 ([M+H]⁺).

Alternatively, Compound 204 was prepared according to Scheme 8 as described below.

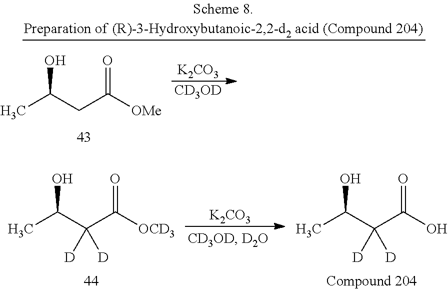

Step 1. Methyl (R)-3-hydroxybutanoate-2,2-d₂ (44): A solution of 43 (1.0 g, 8.46 mmol, [α]20/D −25±1°, c=6% in H₂O, Sigma Aldrich) in methanol-d₄ (25 mL, 99.8 atom % D, Cambridge Isotopes) was treated with potassium carbonate (0.12 g, 0.85 mmol) and heated to reflux. After 3 days, the reaction mixture was concentrated under reduced pressure, and fresh methanol-d₄ (25 mL) and potassium carbonate (0.12 g, 0.85 mmol) were added to the residue. The mixture was heated at reflux for 16 h. The reaction mixture was concentrated under reduced pressure to give crude 44 (1.0 g) which was used subsequently without further work up or purification.

¹H NMR (CDCl₃, 400 MH_{z}): δ 1.22-1.23 (d, 3H), 2.39-2.49 (m, 2H), 4.15-4.21 (m, 1H).

Step 2. (R)-3-Hydroxybutanoic-2,2-d₂ acid (Compound 204): A solution of 44 (1.0 g, 8.32 mmol) in methanol-d₄ (15 mL, 99.8 atom % D, Cambridge Isotopes) and deuterium oxide (6 mL, 99.9 atom % D, Cambridge Isotopes) was treated with potassium carbonate (9.98 mmol, 1.2 equiv) at room temperature for 3 days. The reaction mixture was concentrated under reduced pressure and diluted with deuterium oxide (20 mL), then extracted with dichloromethane (3×10 mL). The combined organic layers were washed with deuterium oxide (3×10 mL). The combined aqueous layers were acidified with aqueous 1M deuterium chloride (prepared from 35 wt % in D₂O, ≥99 atom % D, Sigma Aldrich and deuterium oxide) to pH ˜6. The crude material was adsorbed onto celite (15 g) and purified by flash chromatography (Interchim system, SorbTech 40 g silica gel column, 45-70% ethyl acetate-hexanes in 35 minutes, then 95% ethyl acetate in hexanes for 40 minutes). (Note the product did not elute from the silica gel). The recovered silica gel was stirred in 2% acetic acid in ethyl acetate, filtered, and the filtrate was concentrated. This material was re-purified by flash chromatography twice (Interchim system, SorbTech 25 g silica gel column, gradient of 45-70% ethyl acetate-hexanes in 35 minutes, then 95% ethyl acetate in hexanes for 40 minutes). The resulting residue was lyophilized from water (5 mL) and acetonitrile (0.1 mL) to give Compound 204 (0.144 g, 16% yield) as a white solid.

¹H NMR (CDCl₃, 500 MHz): δ 1.25-1.27 (d, 3H), 4.21-4.25 (m, 1H), 5.95 (bs, 2H). (6% proton incorporation at each site alpha to the carboxylic acid moiety.) LCMS (method: SorbTech C₁₈ AQ column, 2.1×50 mm; 5-95% acetonitrile/water with 0.1% formic acid in 14 min, with 4 min hold; wavelength: 210 nm): retention time: 0.4 min; (EI-MS): m/z=107.1 ([M+H]⁺).

### Example 7. Evaluation of Metabolic Stability

Materials: β-Hydroxybutyrate Dehydrogenase from Pseudomonas lemoignei, nicotinamide adenine dinucleotide sodium salt (NAD), β-nicotinamide adenine dinucleotide, reduced dipotassium salt (NADH) and Trizma Base were purchased from Sigma-Aldrich.

Determination of Metabolic Stability of Compounds of the Invention Using D-β-hydroxybutyrate Dehydrogenase: 100 mM Tris pH 7.8 was prepared for assessing catalytic activity and as diluent to form corresponding solutions. Enzyme solution was prepared by dissolving lyophilized β-hydroxybutyrate dehydrogenase from Pseudomonas lemoignei in 100 mM Tris pH 7.8 to a final concentration of 200 Units/mL. The enzyme working stock solution (2 Units/mL) was prepared by dilution of stock solution (200 Units/mL) with 100 mM Tris pH 7.8 buffer. 30 mM NAD stock solution was prepared by dissolving NAD in 100 mM Tris pH 7.8 buffer. 80 mM stock solutions of test compounds (Protio D-β-hydroxybutyric acid (DBHB), Compounds 100, 102 and 202) were prepared in 100 mM Tris pH 7.8 buffer. Working stock solutions of test compounds were prepared over the concentration range of 1 to 40 mM by appropriate dilutions in 100 mM Tris pH 7.8 buffer. 10 mM analytical standard stock solution were prepared by dissolving NADH in 100 mM Tris pH 7.8. Analytical standards were prepared over the concentration range of 0.05 to 1.5 mM by appropriate dilutions in 100 mM Tris pH 7.8. Enzymatic assays were conducted by aliquoting 40 μL of working test compound stock solution, 6 μL of enzyme working stock solution, 178 μL 100 mM Tris pH 7.8 into 96-well white clear bottom plates. The enzymatic assay was initiated by adding 16 μL of 30 mM NAD. The reaction was incubated at 37° C. The progress of each reaction was monitored over 15 minutes by measuring absorbance at 340 nM of all wells every minute using a Perkin Elmer Enspire Multimode plate reader. Blank wells were prepared to subtract background. NADH formation was quantified using NADH analytical standard curve on same 96-well plate used to monitor enzyme activity. All reactions were conducted in triplicate.

Data analysis: The activity of β-hydroxybutyrate dehydrogenase from Pseudomonas lemoignei with DBHB, and each of Compounds 100, 102 and 202 as a substrate was assessed using a 96-well plate format. The activity was determined by monitoring the rate of conversion of NAD to NADH by change in absorbance at 340 nm. The appropriate background was subtracted for each concentration and compound investigated and NADH concentration formed was calculated using NADH standard curve. The initial rates (v) for NADH formation as a function of initial test compound concentration (C) to derive Km and Vmax values by non-linear regression using Michaelis-Menten equation using GraphPad Prism 7 for Windows (version 7.03).

$$v = \frac{{V}_{max} \times C}{{K}_{m} + C}$$

Difference between Vmax, Km and catalytic efficiency (Vmax/Km) for each compound was assessed using unpaired t-test with statistical significance using Holm-Sidak method with α=0.05. The unpaired t-test values were calculated using GraphPad Prism 7 for Windows (version 7.03).

Intrinsic clearance (CLᵢₙₜ) and half-life (t_{1/2}) values were calculated for each test compound using the following equations.

$${CL}_{int} = \frac{{V}_{max}}{{K}_{m}}$$

$${t}_{1 \text{/} 2} = \frac{ln ( 2 )}{{CL}_{int}}$$

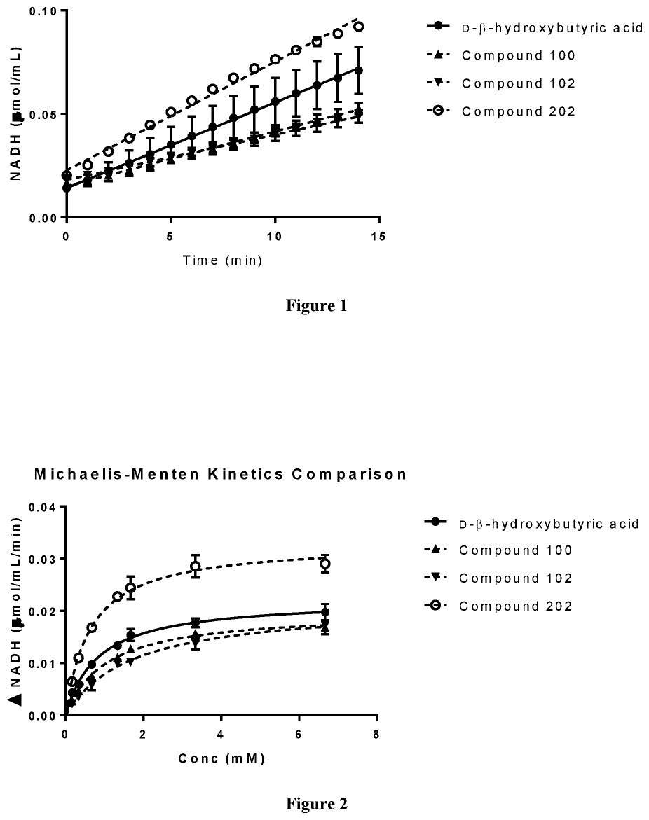

FIGS. 1 and 2 depict graphically the results of this in vitro assay. FIG. 1 shows the formation of NADH as a function of incubation time for each of DBHB, Compound 100, 102 and 202 at a concentration of 0.167 mM. FIG. 2 depicts the substrate saturation plot generated from initial formation rate data for each of DBHB, Compound 100, 102 and 202 over a concentration range of 0.167 mM to 6.7 mM. Table 8, below, shows the results numerically for each compound tested at a concentration of 0.167 mM to 6.7 mM.

TABLE 8
Metabolic Stability of Compounds of the Invention versus D-β-Hydroxybutyric
Acid in D-β-Hydroxybutyrate Dehydrogenase Assay (Km/Vmax Value Comparison)

| | | | | | CLᵢₙₜ | CLᵢₙₜ Ratio | | |
|---|---|---|---|---|---|---|---|---|
| Compound | Vmax (μmol/mL/min) | Km (μmol/mL) | ^{D}V | ^{D}(V/K) | (mL/min) | (NCE/PCE) | T_{1/2} (min) | T_{1/2} Δ (%) |
| D-β-hydroxybutyric acid | 0.0223 | 0.836 | — | — | 0.0267 | — | 26.0 | — |
| Compound 100 | 0.0200 | 1.05 | 1.1 | 1.4 | 0.0190 | 0.7 | 36.4 | 40.2 |
| Compound 102 | 0.0213 | 1.71 | 1.0 | 2.1 | 0.0125 | 0.5 | 55.7 | 114 |
| Compound 202 | 0.0330 | 0.634 | 0.7 | 0.5 | 0.0520 | 2.0 | 13.3 | −48.7 |

NCE = new chemical entity (Compound 100, 102 or 202); PCE = protio chemical entity (DBHB)
*% Δ = [(deuterated species)-(nondeuterated species)](100)/(nondeuterated species)

Using Vmax/Km to predict clearance, when compared to D-β-hydroxybutyric acid, clearance of Compound 100 is predicted to be reduced by 30%; clearance of Compound 102 is predicted to be reduced by 50%; and clearance of Compound 202 is predicted to be increased by 100%. In line with these clearance calculations, the measured half-lives (T_{1/2}) for each of the compounds tested in the D-β-Hydroxybutyrate Dehydrogenase Assay, as shown in Table 8, reveal an increase in the half-lives (T_{1/2}) for Compounds 100 and 102 of 40% and 114% respectively, while the T_{1/2} for Compound 202 is shown to decrease by nearly 49%.

### Example 8. Evaluation of Metabolic Stability in Human Liver Microsomes

Microsomal Assay: Human liver microsomes (20 mg/mL) are obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) are purchased from Sigma-Aldrich.

Determination of Metabolic Stability: 7.5 mM stock solutions of test compounds are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 μL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 0.5 mg/mL human liver microsomes, 0.25-1.0 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-deuterated counterpart of the compound of Formula I and the positive control, 7-ethoxycoumarin (1 μM). Testing is done in triplicate.

Data analysis: The in vitro t_{1/2}s for test compounds are calculated from the slopes of the linear regression of % parent remaining (ln) vs incubation time relationship.

in vitro t_{1/2}=0.693/k

k=−[slope of linear regression of % parent remaining (ln) vs incubation time]

Data analysis is performed using Microsoft Excel Software.

The relevant teachings of all patents, published applications and references cited herein are incorporated by reference in their entirety.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention.

1. A pharmaceutical composition comprising a compound of Formula Ia: 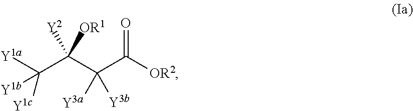
or a pharmaceutically acceptable salt thereof, wherein:
each of Y^{1a}, Y^{1b}, Y^{1c}, Y², Y^{3a} and Y^{3b} is independently H or D;
R¹ is H, D, —C(O)—C₁₋₆ alkyl, or 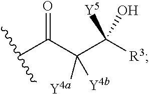
R² is H, D, —C₁₋₆ alkyl, —C₃₋₁₀ cycloalkyl, or 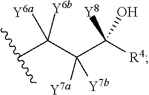
each of Y^{4a}, Y^{4b}, Y⁵, Y^{6a}, Y^{6b}, Y^{7a}, Y^{7b} and Y⁸, when present, is independently H or D; and
each of R³ and R⁴, when present, is independently CH₃ or CD₃;
provided that at least one of Y^{1a}, Y^{1b}, Y^{1c}, Y², Y^{3a} and Y^{3b} is D; and
further provided that when R¹ is H and R² is H, then at least one of Y², Y^{3a} and Y^{3b} is D;
wherein each position designated specifically as deuterium has at least 50.1% incorporation of deuterium;
and a pharmaceutically acceptable carrier.

2. The pharmaceutical composition of claim 1, wherein the compound is a compound of Formula Ib: 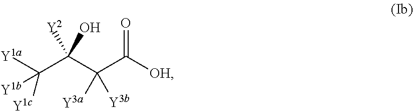
or a pharmaceutically acceptable salt thereof, and wherein
each of Y^{1a}, Y^{1b}, Y^{1c}, Y², Y^{3a} and Y^{3b} is independently H or D;
provided that at least one of Y², Y^{3a} and Y^{3b} is D.

3. The pharmaceutical composition of claim 1, wherein the compound is a compound of Formula 2: 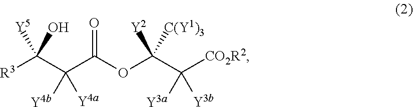
or a pharmaceutically acceptable salt thereof, wherein:
each Y¹ is the same and is H or D;
each of Y², Y^{3a} and Y^{3b} is independently H or D;
each of Y^{4a}, Y^{4b} and Y⁵ is independently H or D;
R² is H or D; and
R³ is CH₃ or CD₃;
provided that at least one of Y¹, Y², Y^{3a} and Y^{3b} is D.

4. The pharmaceutical composition of claim 1, wherein the compound is a compound of Formula 3: 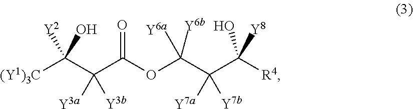
or a pharmaceutically acceptable salt thereof, wherein:
each Y¹ is the same and is H or D;
each of Y², Y^{3a} and Y^{3b} is independently H or D;
each of Y^{6a}, Y^{6b}, Y^{7a}, Y^{7b} and Y⁸ is independently H or D; and
R⁴ is CH₃ or CD₃;
provided that at least one of Y¹, Y², Y^{3a} and Y^{3b} is D.

5. The pharmaceutical composition of claim 1, wherein the compound is a compound of Formula 4: 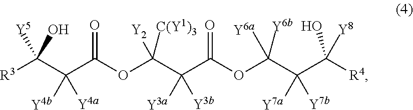
or a pharmaceutically acceptable salt thereof, wherein:
each Y¹ is the same and is H or D;
each of Y², Y^{3a} and Y^{3b} is independently H or D;
each of Y^{4a}, Y^{4b} and Y⁵ is independently H or D;
each of Y^{6a}, Y^{6b}, Y^{7a}, Y^{7b} and Y⁸ is independently H or D;
R³ is CH₃ or CD₃; and
R⁴ is CH₃ or CD₃;
provided that at least one of Y¹, Y², Y^{3a} and Y^{3b} is D.

6. The pharmaceutical composition of claim 1, wherein Y^{1a}, Y^{1b} and Y^{1c} are the same and are each H or each D.

7. The pharmaceutical composition of claim 1, wherein Y^{3a} and Y^{3b} are the same and are each H or each D.

8. The pharmaceutical composition of claim 1, wherein Y² is D.

9. The pharmaceutical composition of claim 1, wherein Y² is H.

10. The pharmaceutical composition of claim 1, wherein each Y¹ is D.

11. The pharmaceutical composition of claim 1, wherein each Y¹ is H.

12. The pharmaceutical composition of claim 1, wherein Y^{3a} and Y^{3b} are each H.

13. The pharmaceutical composition of claim 1, wherein Y^{3a} and Y^{3b} are each D.

14. The pharmaceutical composition of claim 2, wherein Y^{1a}, Y^{1b} and Y^{1c} are the same, Y² is D, and the compound is selected from any one of the compounds set forth in the table below:

| | Each Y^{1a}, | | |
|---|---|---|---|
| Compound # | Y^{1b} and Y^{1c} | Y^{3a} | Y^{3b} |
| 100 | H | H | H |
| 101 | H | H | D |
| 102 | D | H | H |
| 103 | D | D | H |
| 104 | H | D | D |
| 105 | D | D | D |

or a pharmaceutically acceptable salt thereof, wherein any atom not designated as deuterium is present at its natural isotopic abundance.

15. The pharmaceutical composition of claim 2, wherein Y^{1a}, Y^{1b} and Y^{1c} are the same, Y² is H, and the compound is selected from any one of the compounds set forth in the table below:

| | Each Y^{1a}, | | |
|---|---|---|---|
| Compound # | Y^{1b} and Y^{1c} | Y^{3a} | Y^{3b} |
| 201 | H | H | D |
| 203 | D | D | H |
| 204 | H | D | D |
| 205 | D | D | D |

or a pharmaceutically acceptable salt thereof, wherein any atom not designated as deuterium is present at its natural isotopic abundance.

16. The pharmaceutical composition of claim 3, wherein each Y¹ is the same, Y² is D, Y^{3a} and Y^{3b} are the same; Y^{4a} and Y^{4b} are the same, R² is H, and the compound is selected from any one of the compounds set forth the table below:

| Compound # | Each Y¹ | Each Y^{3a}/Y^{3b} | Each Y^{4a}/Y^{4b} | Y⁵ | R³ |
|---|---|---|---|---|---|
| 300 | H | H | H | H | CH₃ |
| 301 | D | H | H | H | CH₃ |
| 302 | H | D | H | H | CH₃ |
| 303 | H | H | D | H | CH₃ |
| 304 | H | H | H | D | CH₃ |
| 305 | H | H | H | H | CD₃ |
| 306 | D | D | H | H | CH₃ |
| 307 | D | H | D | H | CH₃ |
| 308 | D | H | H | D | CH₃ |
| 309 | D | H | H | H | CD₃ |
| 310 | H | D | D | H | CH₃ |
| 311 | H | D | H | D | CH₃ |
| 312 | H | D | H | H | CD₃ |
| 313 | H | H | D | D | CH₃ |
| 314 | H | H | D | H | CD₃ |
| 315 | H | H | H | D | CD₃ |
| 316 | D | D | D | H | CH₃ |
| 317 | D | D | H | D | CH₃ |
| 318 | D | D | H | H | CD₃ |
| 319 | D | H | D | D | CH₃ |
| 320 | D | H | D | H | CD₃ |
| 321 | D | H | H | D | CD₃ |
| 322 | H | D | D | D | CH₃ |
| 323 | H | D | D | H | CD₃ |
| 324 | H | D | H | D | CD₃ |
| 325 | H | H | D | D | CD₃ |
| 326 | D | D | D | D | CH₃ |
| 327 | D | D | D | H | CD₃ |
| 328 | D | D | H | D | CD₃ |
| 329 | D | H | D | D | CD₃ |
| 330 | H | D | D | D | CD₃ |
| 331 | D | D | D | D | CD₃ |

or a pharmaceutically acceptable salt thereof, wherein any atom not designated as deuterium is present at its natural isotopic abundance.

17. The pharmaceutical composition of claim 4, wherein each Y¹ is the same; Y² is D; Y^{3a} and Y^{3b} are the same, Y^{6a}, Y^{6b}, Y^{7a}, and Y^{7b} are the same, and the compound is selected from any one of the compounds set forth the table below:

| | | | Each Y^{6a}/Y^{6b} | | |
|---|---|---|---|---|---|
| Compound # | Each Y¹ | Each Y^{3a}/Y^{3b} | Y^{7a}/Y^{7b} | Y⁸ | R⁴ |
| 500 | H | H | H | H | CH₃ |
| 501 | D | H | H | H | CH₃ |
| 502 | H | D | H | H | CH₃ |
| 503 | H | H | D | H | CH₃ |
| 504 | H | H | H | D | CH₃ |
| 505 | H | H | H | H | CD₃ |
| 506 | D | D | H | H | CH₃ |
| 507 | D | H | D | H | CH₃ |
| 508 | D | H | H | D | CH₃ |
| 509 | D | H | H | H | CD₃ |
| 510 | H | D | D | H | CH₃ |
| 511 | H | D | H | D | CH₃ |
| 512 | H | D | H | H | CD₃ |
| 513 | H | H | D | D | CH₃ |
| 514 | H | H | D | H | CD₃ |
| 515 | H | H | H | D | CD₃ |
| 516 | D | D | D | H | CH₃ |
| 517 | D | D | H | D | CH₃ |
| 518 | D | D | H | H | CD₃ |
| 519 | D | H | D | D | CH₃ |
| 520 | D | H | D | H | CD₃ |
| 521 | D | H | H | D | CD₃ |
| 522 | H | D | D | D | CH₃ |
| 523 | H | D | D | H | CD₃ |
| 524 | H | D | H | D | CD₃ |
| 525 | H | H | D | D | CD₃ |
| 526 | D | D | D | D | CH₃ |
| 527 | D | D | D | H | CD₃ |
| 528 | D | D | H | D | CD₃ |
| 529 | D | H | D | D | CD₃ |
| 530 | H | D | D | D | CD₃ |
| 531 | D | D | D | D | CD₃ |

or a pharmaceutically acceptable salt thereof, wherein any atom not designated as deuterium is present at its natural isotopic abundance.

18. The pharmaceutical composition of claim 1, wherein each position designated specifically as deuterium has at least 90% incorporation of deuterium.

19. The pharmaceutical composition of claim 1, wherein each position designated specifically as deuterium has at least 95% incorporation of deuterium.

20. The pharmaceutical composition of claim 1, wherein any atom not designated as deuterium is present at its natural isotopic abundance.

21. The pharmaceutical composition of claim 1, wherein the compound is at least about 90% stereomerically pure.

22. The pharmaceutical composition of claim 1, wherein the pharmaceutical composition is suitable for oral administration.

23. A method of treating a disease or disorder that is responsive to increased brain-derived neurotrophic factor (BDNF), the method comprising administering to a subject in need thereof an effective amount of a compound of Formula I or a pharmaceutical composition comprising a compound of Formula I: 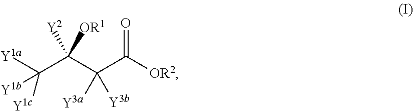
or a pharmaceutically acceptable salt thereof, wherein:
each of Y^{1a}, Y^{1b}, Y^{1c}, Y², Y^{3a} and Y^{3b} is independently H or D;
R¹ is H, D, —C(O)—C₁₋₆ alkyl or 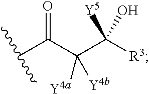
R² is H, D, —C₁₋₆ alkyl, —C₃₋₁₀ cycloalkyl, or 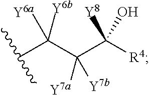
each of Y^{4a}, Y^{4b}, Y⁵, Y^{6a}, Y^{6b}, Y^{7a}, Y^{7b} and Y⁸, when present, is independently H or D;
each of R³ and R⁴, when present, is CH₃ or CD₃;
provided that at least one of Y^{1a}, Y^{1b}, Y^{1c}, Y², Y^{3a} and Y^{3b} is D;
wherein each position designated specifically as deuterium has at least 50.1% incorporation of deuterium;
and a pharmaceutically acceptable carrier.

24. The method of claim 23, wherein the disease or disorder is selected from Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, and eating disorders including anorexia nervosa and bulimia nervosa.

25. A method of treating a disease or disorder that is responsive to increased brain-derived neurotrophic factor (BDNF), the method comprising administering to a subject in need thereof an effective amount of a pharmaceutical composition of claim 1.

26. The method of claim 25, wherein the disease or disorder is selected from Alzheimer's disease, Parkinson's disease, Huntington's disease, Rett syndrome, schizophrenia, major depressive disorder, major depressive disorder with mixed features, bipolar disorder, bipolar mania, bipolar depression, treatment-refractory depression, mild cognitive impairment, cognitive deficits in Parkinson's disease, cognitive deficits in depression, cognition deficits associated with Huntington's disease, subjective cognitive decline, age-related memory loss, a seizure disorder such as epilepsy, generalized anxiety disorder, post-traumatic stress disorder, traumatic brain injury, dementia including Lewy Body Dementia, obsessive-compulsive disorder, and eating disorders including anorexia nervosa and bulimia nervosa.

27. A compound of Formula IIIa: 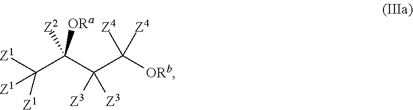
or a pharmaceutically acceptable salt thereof, wherein:
each Z¹ is the same and is H or D;
each Z² is H or D;
each Z³ is the same and is H or D;
each Z⁴ is the same and is H or D;
R^{a} is H or D; and
R^{b} is H or D;
provided that at least one of Z¹, Z², Z³ and Z⁴ is D;
wherein each position designated specifically as deuterium has at least 50.1% incorporation of deuterium;
and a pharmaceutically acceptable carrier.
